SEQUENCE LISTING (9 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 332 aa, Homo sapiens
MLWLFQSLLFVFCFGPGNVVSQSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLRKRRDSLSLSTQRTQGPAESARNLEYVSVSPTNNTVYASVTHSNRETEIWTPRENDTITIYSTINHSKESKPTFSRATALDNVV

SEQ ID NO: 3 - protein, 282 aa, Homo sapiens
MLWLFQSLLFVFCFGPVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLRKRRDSLSLSTQRTQGPESARNLEYVSVSPTNNTVYASVTHSNRETEIWTPRENDTITIYSTINHSKESKPTFSRATALDNVV

SEQ ID NO: 4 - protein, 221 aa, Homo sapiens
MLWLFQSLLFVFCFGPGQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLRKRRDSLSLSTQRTQGPAESARNLEYVSVSPTNNTVYASVTHSNRETEIWTPRENDTITIYSTINHSKESKPTFSRATALDNVV

SEQ ID NO: 5 - protein, 199 aa, Homo sapiens
LMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKM

SEQ ID NO: 6 - protein, 161 aa, Homo sapiens
VPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKM

SEQ ID NO: 7 - DNA, 2754 bp, Homo sapiens
  source 1..2754: mol_type = genomic DNA
agtttatgacagaagggcaaaaacattgactgcctcaaggtctcaagcaccagtcttcaccgcggaaagcatgttgtggctgttccaatcgctcctgtttgtcttctgctttggcccagggaatgtagtttcacaaagcagcttaaccccattgatggtgaacgggattctgggggagtcagtaactcttcccctggagtttcctgcaggagagaaggtcaacttcatcacttggcttttcaatgaaacatctcttgccttcatagtaccccatgaaaccaaaagtccagaaatccacgtgactaatccgaaacagggaaagcgactgaacttcacccagtcctactccctgcaactcagcaacctgaagatggaagacacaggctcttacagagcccagatatccacaaagacctctgcaaagctgtccagttacactctgaggatattaagacaactgaggaacatacaagttaccaatcacagtcagctatttcagaatatgacctgtgagctccatctgacttgctctgtggaggatgcagatgacaatgtctcattcagatgggaggccttgggaaacacactttcaagtcagccaaacctcactgtctcctgggaccccaggatttccagtgaacaggactacacctgcatagcagagaatgctgtcagtaatttatccttctctgtctctgcccagaagctttgcgaagatgttaaaattcaatatacagataccaaaatgattctgtttatggtttctgggatatgcatagtcttcggtttcatcatactgctgttacttgttttgaggaaaagaagagattccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaagttgctgaaaggcctcagaggaattcgggaatgacacgtcttctgatcccatgagacagaacaaagaacaggaagcttggttcctgttgttcctggcaacagaatttgaatatctaggataggatgatcacctccagtccttcggacttaaacctgcctacctgagtcaaacacctaaggataacatcatttccagcatgtggttcaaataatattttccaatccacttcaggccaaaacatgctaaagataacacaccagcacattgactctctctttgataactaagcaaatggaattatggttgacagagagtttatgatccagaagacaaccacttctctccttttagaaagcagcaggattgacttattgagaaataatgcagtgtgttggttacatgtgtagtctctggagttggatgggcccatcctgatacaagttgagcatcccttgtctgaaatgcttgggattagaaatgtttcagatttcaattttttttcagattttggaatatttgcattatatttagcggttgagtatccaaatccaaaaatccaaaattcaaaatgctccaataagcatttcccttgagtttcattgatgtcgatgcagtgctcaaaatctcagattttggagcattttggatattggatttttggatttgggatgctcaacttgtacaatgtttattagacacatctcctgggacatactgcctaaccttttggagccttagtctcccagactgaaaaaggaagaggatggtattacatcagctccattgtttgagccaagaatctaagtcatccctgactccagtgtctttgtcaccaggccctttggactctacctcagaaatatttcttggaccttccacttctcctccaactccttgaccaccatcctgtatccaaccatcaccacctctaacctgaatcctaccttaagatcagaacagttgtcctcacttttgttcttgtccctctccaacccactctccacaagatggccagagtaatgtttttaatataaattggatccttcagtttcctgcttaaaaccctgcaggtttcccaatgcactcagaaagaaatccagtttccatggccctggatggtctggcccacctccagcctcagctagcattacccttctgacactctctatgtagcctccctgatcttctttcagctcctctattaaaggaaaagttctttatgttaattatttacatcttcctgcaggcccttcctctgcctgctggggtcctcctattctttaggtttaattttaaatatgtcacctcctaagagaaaccttcccagaccactctttctaaaatgaatcttctaggctgggcatggtggctcacacctgtaatccctgtactttgggaggccaaggggggagatcacttgaggtcaggagttcaagaccagcctggccaacttggtgaaaccccgtctttactaaaaatacaaaaaaattagccaggcgtggtggtgcacccctaaaatcccagctacttgagagactgaggcaggagaatcgcttgaacccaggaggtggaggttccagtgagccaaaatcatgccaatgtattccagtctgggtgacagagtgagactctgtctcaaaaaataaataaataaaataaaatgaaatagatcttataaaaaaaa

SEQ ID NO: 8 - protein, 190 aa, synthetic construct
  REGION 1..190: note = soluble SLAMF6 polypeptide
MLWLFQSLLFVFCFGPGNVVSVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMGSHHHHHH

SEQ ID NO: 9 - protein, 9 aa, synthetic construct
  REGION 1..9: note = Synthetic peptide
EGSRNQDWL

(19) United States
(12) Patent Application Publication
(10) Pub. No.: US 2026/0151420 A1
(43) Pub. Date: Jun. 4, 2026

(54) COMBINATION CANCER IMMUNOTHERAPY

(71) Applicant: HADASIT MEDICAL RESEARCH SERVICES AND DEVELOPMENT LTD., Jerusalem (IL)

(72) Inventors: Michal Lotem, Reut (IL); Emma Hajaj, Netanya (IL); Galit Eisenberg, Modiin (IL)

(21) Appl. No.: 19/460,857

(22) Filed: Jan. 27, 2026

Related U.S. Application Data
(62) Division of application No. 17/622,225.
(60) Provisional application No. 62/882,625, filed on Aug. 5, 2019.

(30) Foreign Application Priority Data
Jun. 24, 2019 (IL) 267614

(51) Int. Cl. A61K 31/712; A61K 38/17; A61K 39/395; A61K 40/11; A61K 40/32; A61K 40/42; A61P 35/00; C12N 15/113

(52) U.S. Cl. CPC A61K 31/712; A61K 38/1774; A61K 39/3955; A61K 40/11; A61K 40/32; A61K 40/4269; A61P 35/00; C12N 15/1138; A61K 2239/57

(57) ABSTRACT

The invention relates to cancer management, specifically to therapeutic combinations and methods for immunotherapy of tumors and malignancies. More specifically, embodiments of the invention provide compositions, methods, pharmaceutical packages and combined preparations employing the use of a SLAMF6-mediated T cell activator in combination with a LAG3 inhibitor.

### CROSS-REFERENCE TO RELATED APPLICATIONS

This application is a divisional of U.S. application Ser. No. 17/622,225 filed Dec. 22, 2021, which the U.S. National Stage of International Patent Application No. PCT/IL2020/050698, filed on Jun. 23, 2020, which claims the benefit of and priority to U.S. Provisional Patent Application No. 62/882,625, filed on Aug. 5, 2019, and Israel Patent Application No. 267614, filed on Jun. 24, 2019, the contents of each of which are hereby incorporated by reference in their entireties.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 13,982 byte .xml file named “583US-DIV.xml” created on Jan. 22, 2026.

### FIELD OF THE INVENTION

The invention relates to cancer management, specifically to therapeutic combinations and methods for immunotherapy of tumors and malignancies.

### BACKGROUND OF THE INVENTION

Cancer treatment, and in particular immunotherapy-based approaches, is constrained by the functional impairment of T cell-mediated immunity that often takes place in the tumor microenvironment (TME). For example, T cell may enter a state of exhaustion following prolonged inflammation and/or T cell receptor (TCR) exposure to an antigen under certain conditions (e.g. during a chronic viral infection or in the TME). Exhaustion or impairment of CD8+ tumor-infiltrating lymphocytes (TIL) could be detrimental to the therapeutic outcome and in particular of treatment modalities involving immunotherapy. Gene expression profiles of hypofunctional TIL populations have been reported e.g. by Miller et al. (Nat Immunol 2019, 20: 326-336) and Waugh et al. (J Immunol, 2016, 197(4): 1477-1488).

Checkpoint-blockade therapy seeks to reinvigorate T cell response in exhausted or otherwise hypofunctional TIL populations, by targeting inhibitory receptors known as immune checkpoint molecules, which are upregulated by these cells. Targeting immune checkpoints, and primarily programmed death 1 (PD-1)/programmed death ligand 1 (PD-L1), has been demonstrated to be effective in treating advanced malignancies in some patients, and PD-1/PD-L1 inhibitors have been approved by the Food and Drug Administration (FDA) for treating melanoma, non-small cell lung cancer (NSCLC) and other malignancies. However, despite the impressive clinical success of checkpoint blockade therapy in a subset of selected patients, intrinsic or acquired tumor resistance remains a great challenge, leading to low response rate in large-scale use of immune checkpoint inhibitors in solid tumors. The fundamental mechanisms underlying T cell dysfunction in the TME, their modulation by immune checkpoint molecules and immune checkpoint inhibitors, and the complex interrelations between various checkpoint molecules and other receptors or pathways within the TME, remain poorly understood.

Lymphocyte-activation gene 3 (LAG3, also known as CD223) is a cell surface molecule expressed on activated T cells, TIL, regulatory T cells (Tregs), natural killer (NK) cells, B cells and plasmacytoid dendritic cells (DC). It is an immune checkpoint receptor, and was found to exert diverse biologic effects on T cell function. LAG3 belongs to the immunoglobulin superfamily (IgSF) and associates with the CD3/TCR complex. LAG3 interacts with MHC-II to prohibit the binding of the same MHC molecule to TCR and CD4, thus directly hindering TCR signaling in immune response. Aberrant LAG3 expression has been found in a broad spectrum of human tumors such as melanoma, NSCLC, colorectal cancer, breast cancer, hepatocellular carcinoma, follicular lymphoma, head and neck squamous cell carcinoma, which is significantly associated with aggressive tumor progression and clinicopathological characteristics. Over-expression of LAG3 is detected on various TIL, exhibiting significant immune regulatory impacts (Long et al., Genes & Cancer 2018, 9,5-6: 176-189). LAG3 negatively regulates cellular proliferation, activation, and homeostasis of T cells, in a similar fashion to other immune checkpoint molecules, and has been reported to play a role in Treg suppressive function. LAG3 also helps maintain CD8+ T cells in a tolerogenic state and, working with PD-1, helps maintain CD8 exhaustion during chronic viral infection. LAG3 is further known to be involved in the maturation and activation of DC.

Several approaches involving LAG3-targeted immunotherapy are in clinical development. Among these is IMP321, a soluble LAG3-Ig fusion protein which has been investigated in clinical trials since 2006. Three clinical trials in renal cell carcinoma, metastatic breast carcinoma, and melanoma have been completed with moderate success. IMP321 was found to be a systemic antigen presenting cell (APC) activator, to enhance the proliferation of DC, lessen Treg cells immunosuppressive effects, and allow for optimal cross antigen presenting to CD8+ T cells. Another approach is represented by antibodies to LAG3, acting as immune checkpoint inhibitors. Examples are BMS-986016 (relatlimab), LAG525, REGN3767 and TSR-033.

Due to the limited therapeutic success of immune checkpoint inhibitor monotherapy and the development of resistance, combination therapies directed to multiple immune checkpoint molecules are also being considered. However, the response rate remains relatively low. For example, clinical trials are proceeding to evaluate anti-LAG3 antibodies alone or in combination with anti-PD-1 antibodies. Bispecific polypeptides binding PD-1 and LAG-3 such as MGD013 and FS118, are also undergoing clinical trials. WO18020476 and WO2017019894 disclose various immune checkpoint inhibitors which may be used in combination with various other therapies.

In a further attempt to improve treatment efficacy, the use of predictive markers has been considered. Various molecular markers, including marker combinations considering inter alia immune checkpoint molecules, has been suggested to assist in cancer prognosis and in predicting clinical response to various therapies. Such makers and combinations are disclosed e.g. in Ayers et al. (J Clin Invest. 2017; 127(8): 2930-2940) and in WO07045996, WO17075478 and WO18049025.

The SLAM family of receptors (SFR) is a set of eight receptors and one ligand (CD48) expressed on hematopoietic cells. All SLAM family receptors (SFRs) except 2B4 and CD48 are homotypic binders, i.e. they engage a same ectodomain sequence, either in a cis (same cell) or trans (adjacent cell) configuration. Most cell types express 3-5 members of the SLAM family. SFR generate signals via a bi-phasic recruitment mechanism to tyrosines in their cytoplasmic domain, which are designated immunoreceptor tyrosine-based switch motifs (ITSMs).

SLAMF6, also known as Ly108 or NTB-A, is a homotypic SFR expressed on T cells, NK cells, B cells and DC. SLAMF6 has been linked to T cell anchoring to their target cells, and to cytolysis. The precise role of SLAMF6 in healthy T cells is not sufficiently clear. Engagement of SLAMF6 on human T cells can substitute the CD28 co-stimulatory pathway and induce polarization toward a Th1 phenotype. However, CD4-positive T cells from Ly-108 knockout mice (the murine SLAMF6 ortholog) show impairment in IL-4 production, suggesting a role of SLAMF6 in Th2 polarization. The reason for this discrepancy is not fully elucidated. Activation of SLAMF6 on human NK cells stimulates cytotoxicity and proliferation, as well as IFN-7 and TNF-α production.

Valdez et al (J Biol Chem 2004, 279(18), pp. 18662-18669) teach that SLAMF6 activates T cells by homotypic interactions, and specifically enhances Th1 properties. US 2009/017014 to Valdez et al is directed to the PR020080 polypeptide (having an amino acid sequence corresponding to that of canonical SLAMF6), the extracellular portion thereof, homologs, agonists and antagonists thereof, which are suggested as putative modulators of immune diseases. Uzana et al. (J Immunol 2012, 188, pp. 632-640) disclose that SLAMF6 blockade on antigen presenting cells (APC) by specific antibodies inhibited cytokine secretion from CD8+ lymphocytes.

Since SLAMF6 is expressed on certain hematopoietic tumors, vaccination using peptide epitopes derived from this molecule has been proposed, to induce an anti-tumor immune response against tumors aberrantly expressing this antigen. See, e.g., WO 2006/037421. In addition, targeting these epitopes with antibodies or immunotoxin conjugates thereof has been suggested, e.g. in US2011171204. Additional antibodies against SLAMF6 are described, for example, by Krover et al. (British Journal of Haematology 2007, 137, pp. 307-318). These antibodies exerted cytotoxic effects on SLAMF6 expressing lymphocytes, and had no effect on T cell proliferation or cytokine secretion.

EP2083088 discloses a method for treating cancer in a patient comprising modulating the level of an expression product of a gene selected from the group consisting of inter alia SLAMF6 in certain tumor types. The publication discloses that the method is useful for treating a patient characterized by over-expression of said gene.

WO 03/008449 relates to NTB-A polypeptides, nucleic acid molecules encoding the same and uses thereof. The publication also relates to methods of regulating NK cells activity by regulating the activity of NTB-Ain vitro, ex vivo or in vivo, and to methods of screening active compounds using NTB-A or fragments thereof, or nucleic acid encoding the same, or recombinant host cells expressing said polypeptide. Further disclosed is the use of a compound that regulates the activity of an NTB-A polypeptide in the preparation of a medicament to regulate an immune function in a subject.

Yigit et al (Cancer Immunol Res Jul. 17, 2019) suggest a role for SLAMF6 as a regulator of exhausted CD8+ T cells in cancer. In particular, the publication demonstrates that a SLAMF6-binding antibody reduced the number of T cells expressing PD-1 and various other exhaustion markers (PD-1+CD3+CD44+CD8+ cells) in the spleen, in addition to its direct effect on tumor progression; both effects were suggested to depend mainly on antibody-dependent cellular cytotoxicity (ADCC).

A recent publication by the inventors and colleagues suggests a major role for SLAMF6 in lymphocyte co-modulation, showing that targeting SLAMF6 by its soluble ectodomain yields CD8+ T cell with powerful anti-tumor reactivity that do not need interleukin-2 (IL-2) supplementation, neither in vitro nor in vivo, in a model of adoptive transfer of Pmel-1 T cells to melanoma bearing mice (Eisenberg et al., Cancer Immunol Res; 6(2) 2018). WO 2015/104711, to some of the present inventors, discloses the use of soluble NTB-A polypeptides or agonists thereof for the treatment of cancer patients, for preventing and treating cytopenia in susceptible patients, and for the ex vivo preparation of improved T cell compositions for adoptive cell therapy.

WO 2019/155474, to some of the present inventors, relates to improved therapeutic modalities for cancer immunotherapy involving specifically modulating the expression and/or activity of SLAMF6 splice variants. WO '474 discloses inter alia compositions and methods for cancer therapy, including adoptive T cell transfer therapies, cell vaccines and/or polypeptide-based medicaments. The publication further discloses compositions and methods providing selective augmentation of SLAMF6 variant 3 (SLAMF6var3) expression or activity on T cells and/or tumor cells.

There remains an unmet medical need for additional effective and safe therapeutic modalities for cancer.

### SUMMARY OF THE INVENTION

Embodiments of the invention relate to compositions, methods, pharmaceutical packages and combined preparations for treatment of cancer. The compositions, methods, pharmaceutical packages and combined preparations according to these embodiments employ the use of a therapeutic combination of a SLAMF6-mediated T cell activator with a LAG3 inhibitor. According to advantageous embodiments, the use of an isolated SLAMF6 ectodomain or an analog thereof in combination with a LAG3 blocking antibody or an analog thereof is contemplated.

The invention is based, in part, on the surprising discovery, that Pmel-1 TCR transgenic mice in which the murine SLAMF6 counterpart, Ly108, has been knocked-out (Pmel-1×Ly108 KO mice) demonstrate differential expression modulation of immune checkpoint molecules. Specifically, SLAMF6 down-regulation was shown to be associated with selective up-regulation of LAG3 expression on Pmel-1×Ly108 KO splenocytes following prolonged antigen-induced activation. In contradistinction, the expression of PD-1, CD244 (SLAMF4) and TIM-3 remained substantially unaltered in SLAMF6-deficient cells.

The invention is further based, in part, on the unexpected finding that activation of Pmel-1×Ly108 KO splenocytes in the presence of LAG3 blocking antibodies results in improved cytokine secretion in the presence of cognate melanoma cells, whereas LAG3 blockade had no effect on Ly108-expressing Pmel-1 splenocytes. Surprisingly, Ly108 down-regulation together with LAG3 blockade resulted in a 3-times higher IFN-7 production compared to the Ly108-expressing cells (either with or without LAG3 blockade). These finding were further supported in a melanoma model in vivo, in which mice treated by the combination of SLAMF6 deficient T cells and anti-LAG-3 antibody showed faster reduction and disappearance of their tumors. Overall, the results demonstrate a synergistic effect for targeting SLAMF6 and LAG3 in combination, in augmenting tumor-specific T cell response.

Accordingly, the invention relates in some embodiments to a therapeutic combination of a SLAMF6-mediated T cell activator and a LAG3 inhibitor.

SLAMF6-mediated T cell activators to be used in embodiments of the invention may include soluble SLAMF6 ectodomain polypeptides, including isolated ectodomains of SLAMF6 isoforms (e.g. SLAMF6 splice variants 1-4), and other SLAMF6 ligands (e.g. antibodies) that are functional mimetics (analogs) thereof, capable of enhancing T cell activation or exhibiting other immune-enhancing biological activities mediated by an isolated SLAMF6 ectodomain, as disclosed herein. For example, an isolated SLAMF6 ectodomain is capable of rescuing CD8+ T cells from activation-induced cell death (AICD) and from ionizing radiation, improving interferon gamma (IFN-7) production, enhancing surface CD137 (4-1BB) expression, reversing T cell apoptosis and enhancing tumor cell killing by T cells.

LAG3 inhibitors to be used in embodiments of the invention may include LAG3-specific neutralizing (blocking) antibodies, and other LAG3 ligands having LAG3 antagonistic activity, including, but not limited to soluble LAG3 polypeptides (e.g. soluble LAG3-Ig fusion proteins). For example, without limitation, LAG3 inhibitors may include IMP321, BMS-986016 (relatlimab), LAG525, REGN3767, TSR-033 and combinations thereof. In other embodiments, the use of small molecules and nucleic acid agents, such as small molecule LAG3 inhibitors and LAG3-downregulating nucleic acids (e.g. antisense or siRNA oligonucleotides) is contemplated.

In some embodiments, the therapeutic combination is formulated in the form of a pharmaceutical composition, optionally further comprising one or more carriers, excipients or diluents. In other embodiments the therapeutic combination is provided in the form of a first pharmaceutical composition comprising the SLAMF6-mediated T cell activator and a second pharmaceutical composition comprising the LAG3 inhibitor. In yet further embodiments, the therapeutic combination is provided in the form of a bispecific therapeutic agent, comprising a SLAMF6-mediated T cell activator covalently linked to a LAG3 inhibitor (e.g. a polypeptide comprising a SLAMF6 ectodomain fused to a soluble LAG3 polypeptide or a LAG3-binding fragment of a LAG3-neutralizing antibody), which may optionally be formulated as a pharmaceutical composition. In other embodiments, the combination includes a plurality of SLAMF6-mediated T cell activators and/or LAG3 inhibitors, e.g. one or more SLAMF6-mediated T cell activators as disclosed herein in combination with one or more LAG3 inhibitors as disclosed herein. The SLAMF6-mediated T cell activator and the LAG3 inhibitor are provided in therapeutically effective amounts, namely in amounts that are effective, when used in combination, to enhance an anti-tumor immune response or to otherwise exert a beneficial cancer-ameliorating effect. These combinations are collectively referred to herein as the therapeutic combinations of the invention, wherein each possibility represents a separate embodiment of the invention.

Thus, the invention provides in one aspect a pharmaceutical composition, comprising therapeutically effective amounts of a SLAMF6-mediated T cell activator and a LAG3 inhibitor as the sole active ingredients, and optionally one or more carriers, excipients or diluents. In some embodiments, the pharmaceutical composition may be formulated for systemic parenteral administration or for intratumoral administration. In certain particular embodiments, said pharmaceutical composition is formulated for intravenous or subcutaneous administration. In certain embodiments the SLAMF6-mediated T cell activator is selected from the group consisting of soluble SLAMF6 ectodomain polypeptides, anti-SLAMF6 antibodies, and analogs thereof. In certain embodiments the LAG3 inhibitor is selected from the group consisting of soluble LAG3 polypeptides, anti-LAG3 antibodies, and analogs thereof.

In another aspect, there is provided a pharmaceutical pack, comprising a first pharmaceutical composition comprising the SLAMF6-mediated T cell activator and a second pharmaceutical composition comprising the LAG3 inhibitor in therapeutically effective amounts. In some embodiments, the pharmaceutical pack further comprises instructions for use, e.g. for administering the first pharmaceutical composition and the second pharmaceutical composition in concurrent or sequential combination in the treatment of cancer. In another embodiment, said pharmaceutical pack comprises said SLAMF6-mediated T cell activator and said LAG3 inhibitor as the sole active ingredients.

In various embodiments, the combination may be used therapeutically, e.g. in cancer management. For example, the invention relates in some embodiments to a therapeutic combination of the invention for use in the treatment of cancer, or in inducing or enhancing anti-tumor immunity.

In another aspect there is provided a method of treating a tumor in a subject in need thereof, comprising administering to the subject therapeutically effective amounts of a SLAMF6-mediated T cell activator and a LAG3 inhibitor in concurrent or sequential combination, thereby treating the tumor in said subject.

In another aspect there is provided a method for enhancing an anti-tumor immune response in a subject in need thereof, comprising administering to the subject therapeutically effective amounts of a SLAMF6-mediated T cell activator and a LAG3 inhibitor in concurrent or sequential combination, thereby enhancing the anti-tumor immune response in said subject. In one embodiment, the anti-tumor immune response is a T-cell mediated response.

In certain embodiments the SLAMF6-mediated T cell activator is selected from the group consisting of soluble SLAMF6 ectodomain polypeptides, anti-SLAMF6 antibodies, and analogs thereof. In certain embodiments the LAG3 inhibitor is selected from the group consisting of soluble LAG3 polypeptides, anti-LAG3 antibodies, and analogs thereof.

In various embodiments of the methods of the invention, the cancer or tumor may be a melanoma or another solid tumor, e.g. of the skin, urinary tract, head and neck, brain, bladder, liver, lung, breast, reproductive organs (e.g. ovary, cervix), prostate, intestinal tract (e.g. colon), bone, and connective tissues, wherein each possibility represents a separate embodiment of the invention. In other embodiments (e.g. when ex-vivo administration is contemplated, for example in the preparation of cell compositions adapted for adoptive transfer immunotherapy as detailed below), the treatment of hematological malignancies is further contemplated.

In certain embodiments, particularly advantageous tumors to be treated by the methods of the invention are solid tumors positive for one or more molecular markers selected from the group consisting of MHC class II, LAG3, CD4 and CD8, wherein each possibility represents a separate embodiment of the invention. As used herein, a tumor positive for a molecular marker is a tumor in which the marker is found on at least 1% of the cells within the tumor microenvironment (TME), for example of the cells collected from a tumor biopsy. In various embodiments, the tumor to be treated is characterized by expression of MHC class II, LAG3, CD4 and/or CD8 on the surface of at least 1%, at least 2%, at least 3% or at least 5% of the TME total cell count. In other embodiment, the tumor to be treated is characterized by expression of MHC class II, LAG3, CD4 and/or CD8 on the surface of at least 1%, at least 5%, at least 10% or at least 25% of the tumor-infiltrating lymphocytes (TIL). In other particular embodiments said tumor is positive for 2, 3 or 4 of said molecular markers. Each possibility represents a separate embodiment of the invention.

In another embodiment, the methods of the invention are advantageously useful even in the treatment of tumors that are resistant to, or otherwise not amenable for treatment with, other immunotherapies, including, but not limited to, immune checkpoint inhibitors and hitherto known combinations thereof. In various embodiments, the subject is afflicted with a tumor resistant to, or otherwise not amenable for treatment with, LAG3 inhibitors, PD-1/PD-L1 inhibitors, CTLA4 inhibitors, or combinations thereof, wherein each possibility represents a separate embodiment of the invention. In another embodiment, the tumor to be treated does not over-express PD-1 and/or PD-L1. In another embodiment, said tumor is negative for PD-1 and/or PD-L1. In yet another embodiment, said tumor is characterized by surface expression of PD-1 and/or PD-L1 on less than 1%, 5%, or 10% of the TIL.

The therapeutic combinations of the invention may optionally be administered with other cancer therapies, e.g. immunotherapies. In certain embodiments, the therapeutic combinations of the invention may be administered to a subject in need thereof in concurrent or sequential combination with adoptive transfer cell therapy. For example, adoptive transfer cell therapy protocols typically include a step of lympho-depletion about a week before administration of the adoptive transfer cell composition, to yield a favorable condition for infused cell persistence. In various embodiments, the therapeutic combinations of the invention may be administered to the subject prior to cell transplantation, e.g. prior to, concomitantly with, or following a lympho-depleting preparative regimen. In another embodiment, the therapeutic combinations of the invention may be effective in pre-conditioning the subject to receiving an adoptive transfer cell composition, such that the need for conventional lympho-depleting agents may be reduced or even eliminated. For example, the therapeutic combinations of the invention may be administered to the subject (typically a subject afflicted with a solid tumor) for 3-14 days prior to administering said subject with an adoptive transfer cell composition.

However, the therapeutic combinations of the invention are also remarkably effective when administered as sole active ingredients (in the absence of additional anti-cancer agents). In certain embodiments, the subject to be treated by the methods of the invention is not under treatment regimen with other immune checkpoint inhibitors, such as PD-1/PD-L1 inhibitors and/or CTLA4 inhibitors. In another embodiment the subject is afflicted with a tumor resistant to immune checkpoint inhibitor therapy selected from the group consisting of LAG3 inhibitors, PD-1 inhibitors, PD-L1 inhibitors, CTLA4 inhibitors, and combinations thereof.

In another aspect there is provided a method for preparing a cell composition adapted for adoptive transfer immunotherapy, comprising the step of ex vivo administering to an immune cell population a SLAMF6-mediated T cell activator and a LAG3 inhibitor. In various embodiments, the cell population may contain T cells, NK cells or combinations thereof. In another embodiment, said cell population is expanded in the presence of the SLAMF6-mediated T cell activator and the LAG3 inhibitor. In a particular embodiment, the composition is an adoptive transfer T cell composition.

Thus, in another embodiment the invention relates to a method for preparing a cell composition adapted for adoptive transfer immunotherapy, comprising the step of expanding an immune cell population in the presence of a SLAMF6-mediated T cell activator and a LAG3 inhibitor. In another embodiment the cell population contains T cells, NK cells or combinations thereof. In certain embodiments the SLAMF6-mediated T cell activator is selected from the group consisting of soluble SLAMF6 ectodomain polypeptides, anti-SLAMF6 antibodies, and analogs thereof. In certain embodiments the LAG3 inhibitor is selected from the group consisting of soluble LAG3 polypeptides, anti-LAG3 antibodies, and analogs thereof.

In another aspect the invention relates to a cell composition adapted for adoptive transfer immunotherapy prepared by the method disclosed herein. Thus, provided is a cell composition adapted for adoptive transfer immunotherapy prepared by a method comprising the step of expanding an immune cell population in the presence of a SLAMF6-mediated T cell activator and a LAG3 inhibitor. The resulting cell compositions produced by the methods disclosed herein are collectively referred to as the adoptive transfer cell compositions of the invention.

In another aspect the invention relates to a cell composition adapted for adoptive transfer immunotherapy prepared by the method disclosed herein, for use in the treatment of cancer, or in inducing or enhancing anti-tumor immunity. According to additional embodiments the invention relates to methods of treating a tumor, or for enhancing an anti-tumor immune response in a subject in need thereof, comprising administering to the subject an adoptive transfer cell composition of the invention. Preferably, the adoptive transfer cell compositions of the invention are histocompatible with, or autologous to, the subject to be treated. In various embodiments, the subjects to be treated by the adoptive transfer cell compositions of the invention are as described herein with respect to subjects to be treated by the therapeutic combinations of the invention, wherein each possibility represents a separate embodiment of the invention.

Thus, in certain embodiments, the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment the method further comprises administering a SLAMF6-mediated T cell activator and a LAG3 inhibitor in concurrent or sequential combination with the cell composition adapted for adoptive transfer. In another embodiment the subject is not under treatment regimen with other immune checkpoint inhibitors.

Other objects, features and advantages of the present invention will become clear from the following description and drawings.

### BRIEF DESCRIPTION OF THE DRAWINGS

FIG. 1. is a scheme illustrating the generation of the Pmel-1×Ly108 KO mice.

FIG. 2. demonstrates that SLAMF6 down-regulation is associated with selective up-regulation of LAG3 expression following T cell activation.

FIG. 3. shows a synergistic effect of SLAMF6 and LAG3 in combination in augmenting tumor-specific T cell response. Left—Pmel-1 cells; right—Pmel-1×Ly108 KO cells. * indicates P<0.05.

FIGS. 4A-4C. demonstrate improved anti-tumor efficacy in a melanoma model in vivo by targeting SLAMF6 and LAG3 in combination. FIG. 4A presents a scheme of the experimental layout. FIG. 4B. shows tumor volume (Mean±SEM) until day 30 post tumor inoculation. FIG. 4C. shows tumor volume on day 16 post tumor inoculation. *, P<0.05, **, P<0.01.

FIG. 5. demonstrates tumor growth in vivo in melanoma bearing mice after adoptive therapy with Pmel-1 splenocytes expressing native SLAMF6 supplemented with systemic administration of an anti LAG3 antibody (anti-LAG3), compared to control mice in the absence of any treatment (No ACT). Tumor volume measurements (Mean±SEM) are shown from day 7 to day 58 post tumor inoculation.

FIG. 6. shows a synergistic effect of a SLAMF6-mediated T cell activator and a LAG3 inhibitor in augmenting tumor-specific T cell response. A soluble SLAMF6 ectodomain polypeptide corresponding to SLAMF6var3 (seSLAMF6 V3), an anti LAG3 antibody (anti Lag3) or a combination of these agents were supplemented to Pmel-1 splenocytes during activation. Cells activated in the absence of supplementation (No treatment) served as a control. IFN-7 secretion was measured after co-culture of the activated splenocyte with cognate melanoma cells (IFN-g). ***, P=0.0002.

### DETAILED DESCRIPTION OF THE INVENTION

The invention relates to cancer management, specifically to therapeutic combinations and methods for immunotherapy of tumors and malignancies. More specifically, embodiments of the invention provide compositions, methods, pharmaceutical packages and combined preparations employing the use of a SLAMF6-mediated T cell activator in combination with a LAG3 inhibitor.

The invention is based, in part, on the surprising discovery of a specific interrelation or synergism that may be exerted by targeting SLAMF6 and LAG3 in combination, thereby providing unexpectedly improved tumor-specific T cell response.

The invention relates in some embodiments to a therapeutic combination of a SLAMF6-mediated T cell activator and a LAG3 inhibitor, also referred to in some embodiments as a synergistic combination.

In one aspect there is provided a pharmaceutical composition comprising a therapeutic combination of the invention, and optionally one or more carriers, excipients or diluents.

In another aspect there is provided a pharmaceutical pack, comprising a therapeutic combination of the invention.

In another aspect there is provided a method of treating cancer in a subject in need thereof, comprising administering to the subject a therapeutic combination of the invention, thereby treating cancer in said subject.

In another aspect there is provided a method for enhancing an anti-tumor immune response in a subject in need thereof, comprising administering to the subject a therapeutic combination of the invention, thereby enhancing the anti-tumor immune response in said subject.

In another aspect the invention provides a method for preparing a cell composition adapted for adoptive transfer immunotherapy, comprising incubating an immune cell population with a therapeutic combination of the invention.

In another aspect the invention provides a cell composition adapted for adoptive transfer immunotherapy, prepared by a method comprising incubating an immune cell population with a therapeutic combination of the invention.

In another aspect the invention relates to methods for of treating a tumor, or for enhancing an anti-tumor immune response in a subject in need thereof, comprising administering to the subject an adoptive transfer cell composition prepared by a method comprising incubating an immune cell population with a therapeutic combination of the invention.

### Combinations

In various embodiments, SLAMF6-mediated T cell activators and/or LAG3 inhibitors that may be used as active agents in the compositions, methods, and combinations of the invention may include antibodies, polypeptide ligands (e.g. isolated SLAMF6 ectodomains or soluble LAG3 polypeptides), small molecules, nucleic acid agents (e.g. antisense oligonucleotides), and conjugates or combinations thereof. Such active agents may be synthesized or produced recombinantly using methods known in the art. In advantageous embodiments, the therapeutic combinations of the invention are used as the sole active ingredients. In some embodiments, the active agents may be further conjugated to or fused with additional exogenous non-active agents, such as plasma half-life elongating moieties. In particular, the use of additional immune checkpoint inhibitors, such as PD-1 or PD-L1 inhibitors, is explicitly excluded from advantageous embodiments of the invention.

Polypeptides and peptides may conveniently be produced by recombinant technology. Recombinant methods for designing, expressing and purifying proteins and peptides are known in the art (see, e.g. Sambrook et al., 2001, Molecular Cloning: A Laboratory Manual, Cold Springs Harbor Laboratory, New York). Nucleic acid molecules may include DNA, RNA, or derivatives of either DNA or RNA. An isolated nucleic acid sequence encoding a polypeptide or peptide can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, modified nucleic acid sequences in which nucleotides have been inserted, deleted, substituted, and/or inverted in such a manner that such modifications do not substantially interfere with the nucleic acid molecule's ability to encode a functional product. Apolynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. Polynucleotides that include more or less nucleotides can result in the same or equivalent proteins. Using recombinant production methods, selected host cells, e.g. of a microorganism such as E. coli or yeast, are transformed with a hybrid viral or plasmid DNA vector including a specific DNA sequence coding for the polypeptide and the polypeptide is synthesized in the host upon transcription and translation of the DNA sequence.

The terms “antibody” or “antibodies” as used herein refer to an antibody, preferably a monoclonal antibody, or fragments thereof, including, but not limited to, a full length antibody having a human immunoglobulin constant region, a monoclonal IgG, a single chain antibody, a humanized monoclonal antibody, an F(ab′)2 fragment, an F(ab) fragment, an Fv fragment, a labeled antibody, an immobilized antibody and an antibody conjugated with a heterologous compound. Each possibility represents a separate embodiment of the invention. In one embodiment, the antibody is a monoclonal antibody (mAb). In another embodiment, the antibody is a polyclonal antibody. In another embodiment, the antibody is a humanized antibody.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g. Current Protocols in Immunology, Colligan et al (Eds.), John Wiley & Sons, Inc. (1992-2000), Chapter 17, Section 17.1).

LAG3 belongs to Ig superfamily and contains four extracellular Ig-like domains, which are followed by a transmembrane helical domain and a cytoplasmic domain. The precursor polypeptide also contains an N′ signal peptide. For example, the precursor sequence of human LAG3 may be found in accession no. P18627., as presented below, in which the signal peptide resides at positions 1-22, the extracellular domain resides at positions 23-450 (wherein the four Ig-like domains reside at positions 37-167, 168-252, 265-343 and 348-419, respectively), the transmembrane domain resides at positions 451-471, and the cytoplasmic domain resides at positions 472-525:

(human LAG3 precursor, P18627, SEQ ID NO: 1)
MWEAQFLGLLELQPLWVAPVKPLQPGAEVPVVWAQEGAPAQLPCSPTIPLQDLSLLRRAGVTWQ
HQPDSGPPAAAPGHPLAPGPHPAAPSSWGPRPRRYTVLSVGPGGLRSGRLPLQPRVQLDERGRQ
RGDFSLWLRPARRADAGEYRAAVHLRDRALSCRLRLRLGQASMTASPPGSLRASDWVILNCSFS
RPDRPASVHWFRNRGQGRVPVRESPHHHLAESFLFLPQVSPMDSGPWGCILTYRDGFNVSIMYN
LTVLGLEPPTPLTVYAGAGSRVGLPCRLPAGVGTRSFLTAKWTPPGGGPDLLVTGDNGDFTLRL
EDVSQAQAGTYTCHIHLQEQQLNATVTLAIITVTPKSFGSPGSLGKLLCEVTPVSGQERFVWSS
LDTPSQRSFSGPWLEAQEAQLLSQPWQCQLYQGERLLGAAVYFTELSSPGAQRSGRAPGALPAG
HLLLFLILGVLSLLLLVTGAFGFHLWRRQWRPRRFSALEQGIHPPQAQSKIEELEQEPEPEPEP
EPEPEPEPEPEQL.

Commercially available LAG3 inhibitors include e.g. IMP321, BMS-986016 (relatlimab), LAG525, REGN3767, and TSR-033. In particular, Eftilagimod alpha (IMP321) is a soluble LAG3 fusion protein developed by Immutep. Structurally, it is a 160 kDa protein consisting of the four extracellular domains of LAG3 fused to the Fc region of an IgG1 (LAG3-Ig). Relatlimab (BMS-986016) is a mAb directed to human LAG3 developed by Bristol-Myers Squibb. Ieramilimab (LAG525) is a humanized mAb being developed by Novartis. Fianlimab (REGN3767) is a fully human, hinge-stabilized IgG4 mAb that binds with high affinity to LAG3 and blocks this pathway of inhibitory T-cell signaling, developed by Regeneron Pharmaceuticals. TSR-033 is a humanized IgG4 mAb directed against LAG3, developed by AnaptysBio and TESARO. All of these agents are undergoing clinical trials in various experimental settings. Generally, SLAMF6 is comprised of the following domains in the order of N′ to C′:
I. an N-terminal signal peptide;
II. an extracellular portion (ectodomain), comprising two conserved immunoglobulin (Ig)-like motifs: an N′ Ig-like V-type domain (IgV, having a two-layered β-sheet structure, with predominantly neutral, albeit polar, front surfaces), and a C′ Ig-like C2-type domain (IgC2, characterized by an overall β-strand topology and several disulfide bonds);
III. a helical transmembrane domain; and
IV. a topological (cytoplasmic) domain, containing immunoreceptor tyrosine-based switch motifs (ITSMs), which are docking sites for the SH2 domain of SLAM-associated protein (SAP) and the related Ewing's sarcoma-associated transcript. ITSM motifs carry the consensus sequence TxYxxV/I/L that have overlapping specificity for activating and inhibitory binding partners.

In canonical human SLAMF6 (e.g. accession no. Q96DU3, isoform 1), the signal peptide has been identified to be located at positions 1-21 of the transcribed polypeptide, the ectodomain has been identified to be located at positions 22-226 (wherein IgV was located at positions 35-120 and IgC2 at positions 132-209), the transmembrane domain was located at positions 227-247, and the cytoplasmic (intracellular) domain—at positions 248-331. Exon 2 encodes for the amino acids at positions 17-128. The amino acid sequence of human SLAMF6var1 precursor, as well as the corresponding mRNA, are provided in accession no. NM_001184714.1 (SEQ ID NOs: 2 and 7, respectively).

Human SLAMF6var2 differs from SLAMF6var1 by deletion of a single alanine at position 266. Human SLAMF6var3 (precursor, NM_001184715.1, SEQ ID NO: 3) differs from SLAMF6var1 by deletion of amino acids (aa) 17-65 relative to SEQ ID NO: 1. The deletion includes aa 17-21 residing in the signal peptide, and aa 22-65, residing in the ectodomain. Human SLAMF6var4 (precursor, NM_001184716.1, SEQ ID NO: 4) differs from SLAMF6var1 by deletion of aa 18-128.

Exemplary isolated SLAMF6 ectodomain sequences that may be used in embodiments of the invention are as follows:

(SLAMF6var1 ectodomain, SEQ ID NO: 5)
LMVNGILGESVTLPLEFPAGEKVNFTTWLFNETSLAFIVPHETKSPEIHVTNPKQGKRLNFTQS
YSLQLSNLKMEDTGSYRAQISTKTSAKLSSYTLRILRQLRNIQVTNHSQLFQNMTCELHLTCSV
EDADDNVSFRWEALGNTLSSQPNLTVSWDPRISSEQDYTCIAENAVSNLSFSVSAQKLCEDVKI
QYTDTKM.

VPHETKSPEIHVTNPKQGKRLNFTQSYSLQLSNLKMEDTGSYRAQISTKTS AKLSSYTLRILRQLRNIQVTNHSQLFQNMTCELHLTCSVEDADDNVSFRWEALGNTLSS QPNLTVSWDPRISSEQDYTCIAENAVSNLSFSVSAQKLCEDVKIQYTDTKM (SLAMF6var3 ectodomain, SEQ ID NO: 6, which may be used with or without the first 5 N′ amino acids). Conveniently, SLAMF6 ectodomain polypeptides may be used (e.g. produced recombinantly in an animal cell system) with a preceding signal peptide.

As used herein, the term “SLAMF6-mediated T cell activator” relates to a therapeutic agent as disclosed herein, which acts directly on SLAMF6 or a ligand thereof, and is capable of activating T cells as disclosed herein. A “LAG3 inhibitor” as referred to herein is a therapeutic agent which acts directly on LAG3 or a ligand thereof, and is capable of inhibiting LAG3-induced cellular pathways, as detailed herein.

In some embodiments, the SLAMF6-mediated T cell activator to be used in the therapeutic combinations of the invention is selected from the group consisting of soluble SLAMF6 ectodomain polypeptides, anti-SLAMF6 antibodies, and analogs thereof. In some embodiments, the LAG3 inhibitor is selected from the group consisting of soluble LAG3 polypeptides, anti-LAG3 antibodies, and analogs thereof. For example, in various embodiments, the therapeutic combination may include a SLAMF6 ectodomain polypeptide and a soluble LAG3 polypeptide, a SLAMF6 ectodomain polypeptide and an anti-LAG3 antibody, an anti-SLAMF6 antibody and an anti-LAG3 antibody, or an anti-SLAMF6 antibody and a soluble LAG3 polypeptide, wherein each possibility represents a separate embodiment of the invention. In various other embodiments, the use of analogs including functional and structural mimetics of said polypeptides and antibodies, is contemplated.

For example, without limitation, a SLAMF6-mediated T cell activator such as an isolated human SLAMF6 ectodomain is capable of rescuing CD8+ T cells from activation-induced cell death (AICD) and from ionizing radiation, improving interferon gamma (IFN-γ) production, enhancing surface CD137 (4-1BB) expression, reversing T cell apoptosis and enhancing tumor cell killing by T cells. In another example, a LAG3 inhibitor such as Fianlimab is capable of blocking a pathway of LAG3-mediated inhibitory signaling in T cells. In various embodiments, functional mimetics of these agents retain at least 70%, 80%, 90%, 95%, 98% and up to 100% of the activity of said agents. In other embodiments, the term functional mimetic further includes agents exhibiting increased activities, e.g. by 5%, 10%, 20% or 30%, or in other embodiments 40-50%, compared to the SLAMF6-mediated T cell activator or LAG3 inhibitor. In other embodiments, the term functional mimetic further refers to an agent that retains the ligand-binding capacity, namely the ability of the SLAMF6-mediated T cell activator or LAG3 inhibitor to specifically bind the respective target (SLAMF6 or LAG3), thereby exerting the respective biological activities as detailed above.

Further, structural characteristics associated with the above functional characteristics are further disclosed herein, and may be used for preparing suitable analogs that are structural mimetics of particular SLAMF6-mediated T cell activator and LAG3 inhibitors. For example, the extracellular portions of SLAMF6 and LAG3, as well as regions of particular significance within these portions including the Ig-like motifs, have been described. Another well-known example is that the antigen-binding sites of antibodies, and the hypervariable regions thereof including the complementarity-determining regions (CDRs), determine the antigen specificity of the antibody, and may be used for designing antibodies or other molecules having the same specificity. Thus, in advantageous embodiments, a structural mimetic of a molecule as described herein may contain at least the extracellular portion or Ig-like domains of a SLAMF6 polypeptide (optionally lacking the regions encoded by exon 2) or a LAG3 polypeptide, or at least the antigen-binding site of an antibody directed to SLAMF6 or LAG3, which may be chemically derivatized or conjugated to various drugs, half-life elongating moieties and stabilizing groups known in the art (e.g. PEG, BSA or Fc groups), markers and the like, and retain the functional properties as described above. In other embodiments, the analog may be a structural mimetic of these domains or regions, which are conformationally equivalent thereto and thereby retain their ligand-binding capacity.

In a particular embodiment, an analog to be used in compositions and methods of the invention is a bispecific molecule, directed to both SLAMF6 and LAG3. For example, the bispecific molecule may contain genetically fused or chemically conjugated structural moieties associated with the above-specified functions with respect to SLAMF6 and LAG3, including, but not limited to: a SLAMF6 ectodomain polypeptide and a soluble LAG3 polypeptide, a SLAMF6 ectodomain polypeptide and an anti-LAG3 antibody, an anti-SLAMF6 antibody and an anti-LAG3 antibody, and an anti-SLAMF6 antibody and a soluble LAG3 polypeptide. Each possibility represents a separate embodiment of the invention.

Construction of such analogs and recombinant agents, including bispecific molecules, may be made by the skilled artisan based on the sequences and domains as described herein. For example, various formats and platforms for producing bispecific antibodies are available, including, but not limited to Ig-like formats (e.g. using chimeric quadromas or the “knob-in hole” approach), or non-Ig-like formats (lacking an Fc region, e.g. chemically linked Fabs or bivalent and trivalent single-chain variable fragments).

As disclosed herein for the first time, a therapeutic combination of the invention exhibits a synergistic activity with respect to lymphocyte function and anti-tumor immunity. For example, as demonstrated herein, lymphocytes in which the activity of both SLAMF6 and LAG3 is modulated, exhibit at least a threefold enhancement in IFN-7 secretion in the presence of cognate melanoma cells following seven days of co-incubation and expansion compared to an anti-LAG3 antibody when used alone, under the experimental settings described in Example 3, and over a 10-fold enhancement under the experimental settings described in Example 5. Further, as can be seen in Example 4, combined targeting of SLAMF6 and LAG3 in an ACT setting resulted in complete tumor regression and early reduction in tumor size compared to targeting SLAMF6 alone, whereas targeting LAG3 alone did not result in tumor regression. Therapeutic combinations in accordance with embodiments of the invention exhibit an enhancement of at least 25% and typically at least 50%, 70% or 100% in anti-tumor activity, e.g. 1.5-12 fold, 1.5-10 fold, 2-10 fold, 2-4 fold, 5-10 fold and typically about 3-10 fold as measured by IFN-7 secretion or about 2-20, typically about 3-10 or in some embodiments up to 100-200 folds, as measured by tumor volume, compared to each agent alone or to the projected combined activity of both agents. Additional methods for measuring anti-tumor activity are known in the art, and include, without limitation, tumor-specific proliferation, lytic degranulation and/or cytokine (e.g. IL-2) secretion. Accordingly, bispecific molecules provided according to embodiments of the invention further exhibit an enhanced anti-tumor activity as discussed herein (e.g. as measured by IFN-7 secretion from murine splenocytes or human T cells upon incubation with cognate tumor cells).

### Pharmaceutical Compositions

In some embodiments, the invention relates to a pharmaceutical composition comprising therapeutically effective amounts of a SLAMF6-mediated T cell activator and a LAG3 inhibitor as the sole active ingredients, and optionally one or more carriers, excipients or diluents. In one embodiment, the SLAMF6-mediated T cell activator is selected from the group consisting of soluble SLAMF6 ectodomain polypeptides, anti-SLAMF6 antibodies, and analogs thereof. In another embodiment, the LAG3 inhibitor is selected from the group consisting of soluble LAG3 polypeptides, anti-LAG3 antibodies, and analogs thereof. In another embodiment the pharmaceutical composition is formulated for systemic parenteral or intratumoral administration.

Pharmaceutical compositions may be in any pharmaceutical form suitable for administration to a patient, including but not limited to solutions, suspensions, lyophilized powders for reconstitution with a suitable vehicle or dilution prior to usage, capsules, tablets, sustained-release formulations and the like. The compositions may comprise a therapeutically effective amount of an agent of the present invention, preferably in purified form, and a pharmaceutical excipient. As used herein, “pharmaceutical excipient” includes solvents, dispersion media, coatings, antibacterial and antifungal agents, isotonic and absorption delaying agents etc. and combinations thereof, which are compatible with pharmaceutical administration. Hereinafter, the phrases “therapeutically acceptable carrier” and “pharmaceutically acceptable carrier”, which may be used interchangeably, refer to a carrier or a diluent that does not cause significant irritation to an organism and does not abrogate the biological activity and properties of the administered compound. The compositions may also contain other active compounds providing supplemental, additional, or enhanced therapeutic functions.

Pharmaceutical compositions according to the invention are typically liquid formulations suitable for injection or infusion. Examples of administration of a pharmaceutical composition include oral ingestion, inhalation, intravenous and continues infusion, intraperitoneal, intramuscular, intracavity, subcutaneous, cutaneous, or transdermal administration. According to certain particular embodiments, the compositions are suitable for intralesional (e.g. intratumoral) administration. In other embodiments, the compositions are suitable for intravenous administration.

For example, saline solutions and aqueous dextrose and glycerol solutions can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Solutions or suspensions used for intravenous administration typically include a carrier such as physiological saline, bacteriostatic water, Cremophor ELTM (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin. Where necessary, the composition may also include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Solutions or suspensions used for intradermal or subcutaneous application typically include at least one of the following components: a sterile diluent such as water, saline solution, fixed oils, polyethylene glycol, glycerin, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

In certain embodiments, polypeptide active agents are prepared with carriers to protect the polypeptide against rapid elimination from the body. Biodegradable polymers (e.g., ethylene vinyl acetate, polyanhydrides, polyglycolic acid, collagen, polyorthoesters, and polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811). In other particular embodiments, liposomes containing PEG moieties or glycolipids may advantageously be used to enhance blood plasma retention and/or to reduce liver uptake.

Continuous as well as intermittent intravenous administration can also be achieved using an implantable or external pump (e.g., INFUSAID pump). The use of such pumps and adjustment of dosing protocols to the required parameters are well within the abilities of the skilled artisan.

In another embodiment, the pharmaceutical composition is for use in treating a tumor, or in enhancing an anti-tumor immune response, in a subject in need thereof.

In other embodiments, the invention relates to a pharmaceutical pack, comprising a first pharmaceutical composition comprising a SLAMF6-mediated T cell activator, and a second pharmaceutical composition comprising a LAG3 inhibitor, in therapeutically effective amounts. In another embodiment the pharmaceutical pack comprises the SLAMF6-mediated T cell activator and a LAG3 inhibitor as the sole active ingredients. In another embodiment the pharmaceutical pack further comprises instructions for administering the first pharmaceutical composition and the second pharmaceutical composition in concurrent or sequential combination for the treatment of cancer. In another embodiment, the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In another embodiment the first and/or second pharmaceutical composition is formulated for systemic parenteral or intratumoral administration. In another embodiment the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment, said tumor is other than melanoma. In another embodiment, said tumor is a solid tumor. In another embodiment, the tumor excludes chronic lymphocytic leukemia (CLL) or other B cell malignancies. In yet another embodiment, the tumor excludes hematopoietic tumors. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment the instructions include administering said SLAMF6-mediated T cell activator and LAG3 inhibitor in concurrent or sequential combination with adoptive transfer cell therapy. In another embodiment the instructions include administering said SLAMF6-mediated T cell activator and said LAG3 inhibitor for 3-14 days prior to administering said subject with an adoptive transfer cell composition. In another embodiment the subject is not under treatment regimen with other immune checkpoint inhibitors. In another embodiment said subject is not under treatment regimen with PD-1 or PD-L1 inhibitors.

### Subjects and Methods

In various embodiments, the invention relates to compositions and methods for the treatment of cancer (which may be used interchangeably with “tumor”), and/or for inducing or enhancing anti-tumor immunity in a subject in need thereof, wherein each possibility represents a separate embodiment of the invention.

In some embodiments, the subjects to be treated by the compositions and methods of the invention is afflicted with cancer, or at risk for developing cancer (e.g. afflicted with a pre-cancerous lesion or diagnosed with a condition associated with high risk for tumor formation). In another embodiment said subject has been diagnosed with cancer. In another embodiment the compositions and methods are used for preventing or delaying the formation of tumor metastasis. Advantageously, said subject is a human subject.

In certain embodiments, particularly advantageous tumors to be treated by the compositions and methods of the invention are solid tumors positive for one or more molecular markers selected from the group consisting of MHC class II, LAG3, CD4 and CD8, wherein each possibility represents a separate embodiment of the invention. As used herein, a tumor positive for a molecular marker is a tumor in which the marker is found on at least 1% of the cells within the tumor microenvironment (TME), for example of the cells collected from a tumor biopsy. The TME typically contains tumor cells, cancer-associated fibroblasts (CAF), endothelial cells, immune cells such as myelomonocytic cells, myeloid-derived suppressor cells (MDSC), and tumor-infiltrating lymphocytes (TIL), extracellular matrix (ECM), and vasculatures, wherein MHC class II, LAG3, CD4 and CD8 would typically be found on immune cells within the TME. In various embodiments, the tumor to be treated is characterized by expression of MHC class II, LAG3, CD4 and/or CD8 on the surface of at least 1%, at least 2%, at least 3% or at least 5% of the TME total cell count. In other embodiment, the tumor to be treated is characterized by expression of MHC class II, LAG3, CD4 and/or CD8 on the surface of at least 1%, at least 5%, at least 10% or at least 25% of the TIL. In other particular embodiments said tumor is positive for 2, 3 or 4 of said molecular markers. Each possibility represents a separate embodiment of the invention.

In another embodiment, the methods of the invention are advantageously useful even in the treatment of tumors that are resistant to, or otherwise not amenable for treatment with, other immunotherapies, including, but not limited to, immune checkpoint inhibitors and hitherto known combinations thereof. In various embodiments, the subject is afflicted with a tumor resistant to, or otherwise not amenable for treatment with, LAG3 inhibitors, PD-1/PD-L1 inhibitors, CTLA4 inhibitors, or combinations thereof, wherein each possibility represents a separate embodiment of the invention. In another embodiment, the tumor to be treated does not over-express PD-1 and/or PD-L1. In another embodiment, said tumor is negative for PD-1 and/or PD-L1. In yet another embodiment, said tumor is characterized by surface expression of PD-1 and/or PD-L1 on less than 1%, 5%, or 10% of the TIL.

Measuring the surface expression of these molecular markers may be readily performed by the skilled artisan using well-known methods such as flow cytometry, immunohistochemistry and the like. In another embodiment, the methods of the invention comprise the step of determining whether the subject is amenable for treatment, comprising measuring the expression of one or more molecular markers selected from the group consisting of MHC class II, LAG3, CD4 and CD8 in a tumor sample of said subject, and determining that said subject is amenable for treatment if said sample is positive for one or more molecular markers selected from the group consisting of MHC class II, LAG3, CD4 and CD8. In another embodiment there is provided a method for determining whether a subject is amenable for treatment by the compositions and methods of the invention, comprising measuring the expression of one or more molecular markers selected from the group consisting of MHC class II, LAG3, CD4 and CD8 in a tumor sample of said subject, and determining that said subject is amenable for treatment if said sample is positive for one or more molecular markers selected from the group consisting of MHC class II, LAG3, CD4 and CD8. In some embodiments, a tumor determined to be amenable for treatment is characterized by expression of MHC class II, LAG3, CD4 and/or CD8 on the surface of at least 1%, at least 2%, at least 3% or at least 5% of the TME total cell count. In other embodiment, the tumor determined to be amenable for treatment is characterized by expression of MHC class II, LAG3, CD4 and/or CD8 on the surface of at least 1%, at least 5%, at least 10% or at least 25% of the TIL. In other particular embodiments said tumor is positive for 2, 3 or 4 of said molecular markers. Each possibility represents a separate embodiment of the invention.

In other embodiments, the invention relates to a method of treating a tumor in a subject in need thereof, comprising administering to the subject therapeutically effective amounts of a SLAMF6-mediated T cell activator and a LAG3 inhibitor in concurrent or sequential combination, thereby treating the tumor in said subject. In one embodiment, the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In another embodiment the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment, said tumor is other than melanoma. In another embodiment, said tumor is a solid tumor. In another embodiment, the tumor excludes chronic lymphocytic leukemia (CLL) or other B cell malignancies. In yet another embodiment, the tumor excludes hematopoietic tumors. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment the SLAMF6-mediated T cell activator and a LAG3 inhibitor are administered in concurrent or sequential combination with adoptive transfer cell therapy. In another embodiment said SLAMF6-mediated T cell activator and said LAG3 inhibitor are administered to the subject for 3-14 days prior to administering said subject with an adoptive transfer cell composition. In another embodiment the subject is not under treatment regimen with other immune checkpoint inhibitors. In another embodiment said subject is not under treatment regimen with PD-1 or PD-L1 inhibitors. In another embodiment, the SLAMF6-mediated T cell activator and a LAG3 inhibitor are administered to said subject intralesionally or by systemic parenteral administration. In certain particular embodiments, the SLAMF6-mediated T cell activator and a LAG3 inhibitor are administered to said subject intratumorally, intravenously or subcutaneously. In another particular embodiment, the SLAMF6-mediated T cell activator and a LAG3 inhibitor are administered to said subject intraperitoneally. In another particular embodiment, the SLAMF6-mediated T cell activator and a LAG3 inhibitor are not administered to said subject intraperitoneally.

In other embodiments, the invention provides a method for enhancing an anti-tumor immune response in a subject in need thereof, comprising administering to the subject therapeutically effective amounts of a SLAMF6-mediated T cell activator and a LAG3 inhibitor in concurrent or sequential combination, thereby enhancing the anti-tumor immune response in said subject. In one embodiment, the anti-tumor immune response is a T-cell mediated response.

As used herein, the term “T-cell mediated response” (or “T-cell mediated anti-tumor immune response”) refers to a tumor-specific adaptive immune response, in which the activity of T cells, and in particular cytotoxic T cells (CD8+) and/or helper T cells (CD4+), exert beneficial effects that reduce, postpone or reverse tumor formation or development and/or alleviate one or more symptoms of cancer in a subject.

In another embodiment of the methods of enhancing an anti-tumor immune response, the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In another embodiment the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment, said tumor is other than melanoma. In another embodiment, said tumor is a solid tumor. In another embodiment, the tumor excludes chronic lymphocytic leukemia (CLL) or other B cell malignancies. In yet another embodiment, the tumor excludes hematopoietic tumors. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment the SLAMF6-mediated T cell activator and a LAG3 inhibitor are administered in concurrent or sequential combination with adoptive transfer cell therapy. In another embodiment said SLAMF6-mediated T cell activator and said LAG3 inhibitor are administered to the subject for 3-14 days prior to administering said subject with an adoptive transfer cell composition. In another embodiment the subject is not under treatment regimen with other immune checkpoint inhibitors. In another embodiment said subject is not under treatment regimen with PD-1 or PD-L1 inhibitors. In another embodiment, the SLAMF6-mediated T cell activator and a LAG3 inhibitor are administered to said subject intralesionally or by systemic parenteral administration. In certain particular embodiments, the SLAMF6-mediated T cell activator and a LAG3 inhibitor are administered to said subject intratumorally, intravenously or subcutaneously. In another particular embodiment, the SLAMF6-mediated T cell activator and a LAG3 inhibitor are administered to said subject intraperitoneally. In another particular embodiment, the SLAMF6-mediated T cell activator and a LAG3 inhibitor are not administered to said subject intraperitoneally.

In another aspect there is provided a therapeutic combination of a SLAMF6-mediated T cell activator and a LAG3 inhibitor for use in treating a tumor in a subject in need thereof. In another embodiment, the combination is for concurrent or sequential use. In another embodiment the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In another embodiment the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment the SLAMF6-mediated T cell activator and a LAG3 inhibitor are for use in concurrent or sequential combination with adoptive transfer cell therapy. In another embodiment the use comprises administering to the subject the therapeutic combination for 3-14 days prior to administering said subject with an adoptive transfer cell composition. In another embodiment the subject is not under treatment regimen with other immune checkpoint inhibitors.

In another aspect there is provided a therapeutic combination of a SLAMF6-mediated T cell activator and a LAG3 inhibitor for use in enhancing an anti-tumor immune response in a subject in need thereof. In another embodiment the anti-tumor immune response is a T-cell mediated response. In another embodiment, the combination is for concurrent or sequential use. In another embodiment the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In another embodiment the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment the SLAMF6-mediated T cell activator and a LAG3 inhibitor are for use in concurrent or sequential combination with adoptive transfer cell therapy. In another embodiment the use comprises administering to the subject the therapeutic combination for 3-14 days prior to administering said subject with an adoptive transfer cell composition. In another embodiment the subject is not under treatment regimen with other immune checkpoint inhibitors.

In some embodiments, the use comprises expanding an immune cell population in the presence of a SLAMF6-mediated T cell activator and a LAG3 inhibitor to thereby provide a cell composition adapted for adoptive transfer immunotherapy, which may be used for treating the tumor or enhancing the anti-tumor immune response in said subject, as disclosed herein.

In various embodiments, the SLAMF6-mediated T cell activator and a LAG3 inhibitor may be administered to a subject in need thereof in concurrent or sequential combination. For example, the SLAMF6-mediated T cell activator may be administered prior to, following, or together with the LAG3 inhibitor, wherein each possibility represents a separate embodiment of the invention.

In various embodiments, treating a tumor may include inducing or enhancing tumor regression, preventing or delaying the onset of symptoms, reducing or reversing the severity of symptoms, reducing tumor size, preventing or delaying the progression of the tumor to a more severe stage or grade, and/or reducing or inhibiting tumor progression. In some embodiments, the compositions and methods of the invention are advantageously used for inducing or enhancing tumor regression. In various embodiments, the compositions and methods of the invention are used for enhancing tumor regression by at least threefold compared to each treatment alone. In other embodiments, the compositions and methods of the invention are used for expediting or shortening the time to achieving measurable anti-tumor response. In various embodiments, the compositions and methods of the invention are used for expediting tumor regression e.g. by at least 2, 4, 6, 8 or 10 days. In yet another embodiment, the compositions and methods of the invention are used for enhancing an anti-tumor immune response by at least about 3-10 folds as measured by IFN-7 secretion or tumor volume, compared to each agent alone and/or to the projected combined activity of both agents. Each possibility represents a separate embodiment of the invention.

### Cell Compositions and Adoptive Transfer Therapy

In another aspect, there is provided a T cell composition prepared as described herein, suitable for adoptive transfer into a recipient subject in need thereof. As used herein, and unless otherwise specified, the term “adoptive transfer” refers to a form of passive immunotherapy where previously sensitized immunologic agents (e.g., cells or serum) are transferred to the recipients. The phrases “adoptive transfer immunotherapy”, “adoptive cell therapy” and “adoptive cell immunotherapy” are used interchangeably herein to denote a therapeutic or prophylactic regimen or modality, in which effector immunocompetent cells, such as the cell compositions of the invention, are administered (adoptively transferred) to a subject in need thereof, to alleviate or ameliorate the development or symptoms of cancer or infectious diseases.

T lymphocytes (T cells) are one of a variety of distinct cell types involved in an immune response. The activity of T cells is regulated by antigen, presented to a T cell in the context of a major histocompatibility complex (MHC) molecule. The T cell receptor (TCR) then binds to the MHC-antigen complex. Once antigen is complexed to MHC, the MHC-antigen complex is bound by a specific TCR on a T cell, thereby altering the activity of that T cell. Proper activation of T lymphocytes by antigen-presenting cells requires stimulation not only of the TCR, but the combined and coordinated engagement of its co-receptors.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response.

Cytotoxic T cells (TC cells, or CTLs) destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells.

Regulatory T cells (Treg cells), formerly known as suppressor T cells, are crucial for the maintenance of immunological tolerance. Their major role is to shut down T cell-mediated immunity toward the end of an immune reaction and to suppress autoreactive T cells that escaped the process of negative selection in the thymus.

The TCR is a complex of integral membrane proteins, wherein stimulation by specific MHC-presented antigen recognition and binding by the clonotype-specific a/P heterodimer leads to activation of transcription and subsequent proliferation and effector functions (such as cytotoxic activity in CD8+ T cells and cytokine secretion in CD4+ T cells). This activation involves other subunits of the receptor complex as detailed below that couple the extracellular liganding event to downstream signaling pathways such as protein phosphorylation, the release of inositol phosphates and the elevation of intracellular calcium levels.

TCR stimulation may be antigen-specific or antigen non-specific (Polyclonal). Suitable antigen-specific TCR activators include antigens bound to MHC molecules, typically in the context of antigen presenting cells (APC). Polyclonal TCR activators are capable of initiating the signal transduction and transcriptional activation pathways associated with specific TCR engagement in the absence of specific antigens. Suitable polyclonal T cell activators include antibodies that bind and crosslink the T cell receptor/CD3 complex, e.g. subunits as described herein. Exemplary antibodies that crosslink the T cell receptor include the HIT3a, UCHT1 and OKT3 monoclonal antibodies. The stimulation is provided at an amount and under conditions as known in the art so as to induce the above-mentioned functional effects.

Typically, compositions for adoptive cell transfer are prepared by methods including activating a T cell population by a TCR stimulation, and expansion of the cells to obtain a therapeutically effective amount of effector T cells for administration. Such methods include but are not limited to, Rapid Expansion Protocols (REP).

In various embodiments, the TCR stimulation may be antigen non-specific (performed, for example, using antibodies specific to CD3 that activate the receptor upon binding, e.g. OKT3) or antigen-specific (using suitable antigen presenting cells and antigen). In the context of cancer treatment, antigen-specific stimulation typically employs stimulation to tumor-associated antigens. The term “tumor-associated antigen” (TAA) refers to any protein, peptide or antigen associated with (carried by, expressed by, produced by, secreted by, etc.) a tumor or tumor cell(s). Tumor-associated antigens may be (nearly) exclusively associated with a tumor or tumor cell(s) and not with healthy normal cells or may be over expressed (e.g., 2 times, 5 times, 10 times, 50 times, 100 times, 1000 times or more) in a tumor tissue or tumor cell(s) compared to healthy normal tissue or cells. More particularly, a TAA is an antigen capable of being presented (in processed form) by MHC determinants of the tumor cell. Hence, tumor-associated antigens are likely to be associated only with tumors or tumor cells expressing MHC molecules. Non-limitative examples of well-known TAA are MART-1, gp100 209-217, gp100 154-163, CSPG4, NY-ESO-1, MAGE-A1, Tyrosinase.

In some embodiments, one commonly used approach for stimulating proliferation, in particular of CD8+ T cells, is the incubation of T cells with soluble anti-CD3 antibody in the presence of Fc receptor-bearing accessory cells (feeder cells), an approach designated the REP. Antibody “presented” to T cells in this manner generates a more effective proliferative signal than soluble anti-CD3 alone or anti-CD3 immobilized on a plastic surface. In the treatment of cancer, adoptive cell therapy typically involves collecting T cells that are found within the tumor of the patient (referred to as tumor-infiltrating lymphocytes, TIL), which are encouraged to multiply ex vivo using high concentrations of IL-2, anti-CD3 and allo-reactive feeder cells. These T cells are then transferred back into the patient along with exogenous administration of IL-2 to further boost their anti-cancer activity.

Thus, according to certain embodiments, activation and/or expansion (e.g. as part of a REP protocol) is performed in the presence of feeder cells. The term “feeder cells” generally refers to cells of one type that are co-cultured with cells of a second type, to provide an environment in which the cells of the second type can be maintained and proliferated. For the purpose of the present invention, this term specifically refers to Fc receptor-bearing accessory cells, which are typically allo-reactive with the T cell containing population to be propagated. In other words, the feeder cells need not be histocompatible with the T-cell containing population to be propagated, and in certain advantageous embodiments the two populations typically HLA-mismatched. Atypical example of feeder cells used in embodiments of the invention is allogeneic normal donor peripheral blood mononuclear cells, PBMC. Typically and advantageously, the use of such feeder cells is performed in conjunction with antigen non-specific TCR stimulation, e.g. by incubation with antigen non-specific stimulating antibodies, as detailed herein.

In another embodiment, adoptive transfer T cell compositions are prepared with irradiated PBMC (incapable of proliferation) as feeder cells. For example, PBMC may conveniently be attenuated by irradiation by exposing the cells to 6000RAD. In another embodiment, adoptive transfer T cell compositions are prepared with artificial antigen presenting entities including antigen presenting cells and inert particles carrying antigens, to provide antigen-specific stimulation.

In various embodiments, T cell expansion may be performed for at least 5 and typically at least 6, 7, or 8 days. Typically, expansion is performed for up to about 16, 15, 14, 13, or 12 days, for example 5-15 days, e.g. 6-12 or more typically 8-15 days. In another embodiment, the population comprises CD8+ T cells. In another embodiment, the T cells are CD8+ T cells. In another embodiment, the cells are further genetically engineered or modified (e.g. to exert a desired antigen specificity). For example, in another embodiment, the cells are lymphocytes (e.g. purified T cells such as CTL) genetically engineered to express a TCR pre-designed to re-direct them against cancer cells or against pathogens (e.g. viruses). By means of a non-limitative example, T cells engineered to express a TCR directed against NY-ESO-1, an antigen expressed on many solid tumors, e.g. synovial sarcoma. In another embodiment, the cells are peripheral blood mononuclear cells genetically engineered to express a chimeric antigen receptor (CAR) to re-direct them against cancer cells or pathogens. For example, without limitation, CAR-T cells targeting CD19 may be used for the treatment of B cell malignancies such as acute lymphoblastic leukemia. In another embodiment, the cells are peripheral blood mononuclear cells genetically engineered to express genes that enhance their biological function. For example, without limitation, such genes may include membrane bound cytokine and cytokine receptor (e.g. IL-2 and IL-2R). In another embodiment the population comprises CD4+ T cells. In another embodiment the population comprises a combination of CD8+ T cells and CD4+ T cells.

In some embodiments, the invention further envisages the use of nucleic acid agents for genetic modification, e.g. to modulate the expression of SLAMF6 and/or LAG3. For instance, the invention exemplifies herein the use of genetically modified lymphocytes, in particular for adoptive cell therapy. By means of non-limitative examples, lymphocytes of a subject (or cells adapted to adoptive transfer therapy) may be manipulated to downregulate the expression of SLAMF6 or an isoform thereof, or of LAG3. In other embodiments, the use of antisense oligonucleotides or siRNA may be employed for eliciting short-term downregulation of these genes. In a particular embodiment, the invention employs the use of SLAMF6 expression-modulating oligonucleotides that induce splice-switching of SLAMF6, enhancing the expression of SLAMF6var3 and/or reducing the expression of SLAMF6var1. Non-limitative examples of such oligonucleotides are expression-modulating oligonucleotide of 15-30 nucleotides in length, specifically hybridizable with a nucleic acid target located at positions 252-271, 257-276 or 250-278 of SEQ ID NO: 7. In another embodiment, said expression-modulating oligonucleotide is not specifically hybridizable with a target located at positions 262-281 of SEQ ID NO: 7. In other embodiments, the use of such nucleic acid agents, and in particular of splice-switching oligonucleotides, is explicitly excluded.

The cell composition may comprise a T cell-containing population in an effective amount. For example, an amount effective for adoptive transfer immunotherapy is an amount sufficient to induce or enhance a beneficial immune response such as an anti-tumor response, e.g. 106 to 1012 cells. It is to be understood, that while cell preparations suitable for in vivo administration, particularly for human subjects, may contain pharmaceutically acceptable excipients or diluents, such preparations are sufficiently devoid of contamination by pathogens, toxins, pyrogens and any other biological and non-biological agents which are not recognized to be pharmaceutically acceptable. For example, without limitation, T cells for adoptive transfer immunotherapy may conveniently be suspended in an injection suitable buffer that contains sterile saline with 2% human albumin, and optionally IL-2 (e.g. 300IU/ml).

According to certain preferable embodiments, the cell composition is histocompatible with the subject to be treated (e.g. autologous cells or MHC II-matched allogeneic cells).

The term “histocompatibility” refers to the similarity of tissue between different individuals. The level of histocompatibility describes how well matched the patient and donor are. The major histocompatibility determinants are the human leukocyte antigens (HLA). HLA typing is performed between the potential donor and the potential recipient to determine how close an HLA match the two are. The term “histocompatible” as used herein refers to embodiments in which all six of the HLA antigens (2 A antigens, 2 B antigens and 2 DR antigens) are the same between the donor and the recipient.

However, in other embodiments, donors and recipients who are “mismatched” at two or more antigens, for example 5 of 6, or in other embodiments, 4 of 6 or 3 of 6 match, may be encompassed by certain embodiments of the invention, despite the donor and recipient not having a complete match. The term “substantially histocompatible” as used herein refers to embodiments in which five out of six of the HLA antigens are the same between the donor and the recipient.

In some embodiments, the invention provides a method for preparing a cell composition adapted for adoptive transfer immunotherapy, comprising the step of expanding an immune cell population in the presence of a SLAMF6-mediated T cell activator and a LAG3 inhibitor. In some embodiments, the cell population contains T cells, NK cells or combinations thereof. In another embodiment the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In some embodiments, expansion may be performed in the presence of accessory cells (e.g. PBMC) and/or cytokines, using methods known in the art. For example, in one embodiment when the composition is an adoptive transfer T cell composition, the expansion is performed by a rapid expansion protocol (REP), e.g. in the presence of irradiated PBMC, anti-CD3 antibodies and IL-2 for 8-15 days. In another embodiment the expansion is performed by providing said cell population with a TCR stimulation and at least one co-stimulation (e.g. for 1-3 weeks, wherein each possibility represents a separate embodiment of the invention). In another embodiment, the expansion is performed by providing said cell population with a TCR stimulation and a SLAMF6-mediated stimulation. In another embodiment, the cell population is a T cell population comprising CD8+ T cells selected from the group consisting of tumor infiltrating leukocytes (TIL), lymph node lymphocytes (lymphocytes within the tumor-draining lymph nodes, LNL), tumor-specific T cell clones, and genetically modified T cells. In another embodiment said cell population expresses a chimeric antigen receptor (CAR). In other embodiments, the cell population may be derived (isolated and/or differentiated) from peripheral blood mononuclear cells (PBMC), or from stem cells including, but not limited to, embryonic stem cells (ESC), hematopoietic stem cells (HSC), mesenchymal stem cells (MSC), umbilical cord blood stem cells (UCB-SC), and induced pluripotent stem cells (iPSC). In another embodiment when the composition is an NK cell composition, expansion may include cultivation of purified NK cells with cytokines such as IL-2 or a combination of IL-12, IL-15 and IL-18, or cultivation of PBMC (which may be CD3 depleted or CD56 enriched) in the presence of e.g. IL-2, IL-15, or IL15+IL-21) for 2-3 weeks. In various embodiments, the immune cell population may be expanded in the presence of said SLAMF6-mediated T cell activator and said LAG3 inhibitor either concurrently or sequentially. For example, in a particular embodiment, said population may be expanded in the presence of said SLAMF6-mediated T cell activator (e.g. isolated SLAMF6 ectodomain) throughout the entire expansion period (e.g. 2-3 weeks), wherein said LAG3 inhibitor (e.g. anti-LAG3 antibody) is supplemented to the expansion culture for the last 1-3 days of the expansion. The resulting cell compositions produced by the methods disclosed herein are collectively referred to as the adoptive transfer cell compositions of the invention, wherein each possibility represents a separate embodiment of the invention.

In other embodiment, the invention provides a cell composition adapted for adoptive transfer immunotherapy prepared by a method comprising the step of expanding an immune cell population in the presence of a SLAMF6-mediated T cell activator and a LAG3 inhibitor. In yet further embodiments, invention relates to methods for treating a tumor, or for enhancing an anti-tumor immune response in a subject in need thereof, comprising administering to the subject the cell composition adapted for adoptive transfer. In another embodiment the cell composition adapted for adoptive transfer is histocompatible with the subject. In another embodiment the cell composition adapted for adoptive transfer is autologous to the subject. In another embodiment the method further comprises administering a SLAMF6-mediated T cell activator and a LAG3 inhibitor in concurrent or sequential combination with the cell composition adapted for adoptive transfer. In another embodiment, the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In another embodiment the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment, said tumor is other than melanoma. In another embodiment, said tumor is a solid tumor. In another embodiment the tumor is a hematopoietic tumor. In another embodiment, the tumor excludes chronic lymphocytic leukemia (CLL) or other B cell malignancies. In yet another embodiment, the tumor excludes hematopoietic tumors. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment said SLAMF6-mediated T cell activator and said LAG3 inhibitor are administered to the subject for 3-14 days prior to administering said subject with the cell composition adapted for adoptive transfer. In another embodiment the subject is not under treatment regimen with other immune checkpoint inhibitors. In another embodiment said subject is not under treatment regimen with PD-1 or PD-L1 inhibitors. In another embodiment, the cell composition adapted for adoptive transfer is administered to said subject intralesionally or by systemic parenteral administration. In certain particular embodiments, the cell composition adapted for adoptive transfer is administered to said subject intratumorally, intravenously or subcutaneously.

In another embodiment there is provided a cell composition adapted for adoptive transfer immunotherapy for use in treating a tumor, or for enhancing an anti-tumor immune response, in a subject in need thereof, wherein the cell composition has been prepared by a method comprising the step of expanding an immune cell population in the presence of a SLAMF6-mediated T cell activator and a LAG3 inhibitor. In another embodiment the cell composition adapted for adoptive transfer is histocompatible with the subject. In another embodiment the cell composition adapted for adoptive transfer is autologous to the subject. In another embodiment the use further comprises administering a SLAMF6-mediated T cell activator and a LAG3 inhibitor in concurrent or sequential combination with the cell composition adapted for adoptive transfer. In another embodiment, the SLAMF6-mediated T cell activator is selected from the group consisting of soluble SLAMF6 ectodomain polypeptides, anti-SLAMF6 antibodies, and analogs thereof. In another embodiment the LAG3 inhibitor is selected from the group consisting of soluble LAG3 polypeptides, anti-LAG3 antibodies, and analogs thereof. In another embodiment the tumor is selected from the group consisting of tumors of the skin, urinary tract, head and neck, brain, bladder, liver, lung, breast, reproductive organs, prostate, intestinal tract, bone, and connective tissues. In another embodiment said tumor is melanoma. In another embodiment, said tumor is other than melanoma. In another embodiment, said tumor is a solid tumor. In another embodiment the tumor is a hematopoietic tumor. In another embodiment, the tumor excludes chronic lymphocytic leukemia (CLL) or other B cell malignancies. In yet another embodiment, the tumor excludes hematopoietic tumors. In another embodiment the tumor is a solid tumor positive for one or more molecular markers selected from the group consisting of MHC class II, LAG3, CD4 and CD8. In another embodiment the tumor is resistant to immune checkpoint inhibitor therapy selected from the group consisting of LAG3 inhibitors, PD-1 inhibitors, PD-L1 inhibitors, CTLA4 inhibitors, and combinations thereof. In another embodiment said SLAMF6-mediated T cell activator and said LAG3 inhibitor are to be administered to the subject for 3-14 days prior to administering said subject with the cell composition adapted for adoptive transfer. In another embodiment the subject is not under treatment regimen with other immune checkpoint inhibitors. In another embodiment said subject is not under treatment regimen with PD-1 or PD-L1 inhibitors. In another embodiment, the cell composition adapted for adoptive transfer is to be administered to said subject intralesionally or by systemic parenteral administration. In certain particular embodiments, the cell composition adapted for adoptive transfer is to be administered to said subject intratumorally, intravenously or subcutaneously.

As disclosed herein, cell compositions prepared using the therapeutic combinations of the invention exhibit improved properties compared to equivalent cell compositions prepared using known methods. In particular, the adoptive transfer cell compositions of the invention exhibit enhanced anti-tumor activity as described herein. In some embodiments, the cell compositions of the invention exhibit an enhancement of at least 25% and typically at least 50%, 70% or 100% in anti-tumor activity, e.g. 2-4 fold and typically about 3-fold as measured by IFN-7 secretion or about 3-10 and up to 100 or 200 fold as measured by tumor volume, compared to equivalent compositions prepared using conventional REP protocols (either with or without the SLAMF6-mediated T cell activator or the LAG3 inhibitor alone). In other embodiments, the expansion in the presence of the therapeutic combination is performed so as to provide an enhancement of at least 25% and typically 2-4-fold (e.g. about 3-fold) in tumor-specific IFN-7 secretion compared to equivalent cells in a conventional REP protocol, e.g. as under conditions as disclosed herein.

As disclosed herein, the therapeutic combinations of the invention may optionally be used in combination with an immunotherapy. In certain embodiments, the therapeutic combinations of the invention may be administered to a subject in need thereof in concurrent or sequential combination with adoptive transfer cell therapy. In a particular embodiment, said adoptive transfer cell therapy is an adoptive transfer cell compositions of the invention.

For example, adoptive transfer cell therapy protocols typically include a step of lympho-depletion about a week before administration of the adoptive transfer cell composition, to yield a favorable condition for infused cell persistence. In various embodiments, the therapeutic combinations of the invention may be administered to the subject prior to cell transplantation, e.g. prior to, concomitantly with, or following a lympho-depleting preparative regimen (e.g. 60 mg/kg cyclophosphamide for 2 days and 25 mg/m2 fludarabine administered for 5 days). In another embodiment, the therapeutic combinations of the invention may be effective in pre-conditioning the subject to receiving an adoptive transfer cell composition, such that the need for conventional lympho-depleting agents may be reduced or even eliminated. For example, the therapeutic combinations of the invention may be administered to the subject (typically a subject afflicted with a solid tumor) for about 3-14 days prior to administering said subject with an adoptive transfer cell composition.

As used throughout the specification herein, and unless indicated otherwise, the term “about” refers to ±10%.

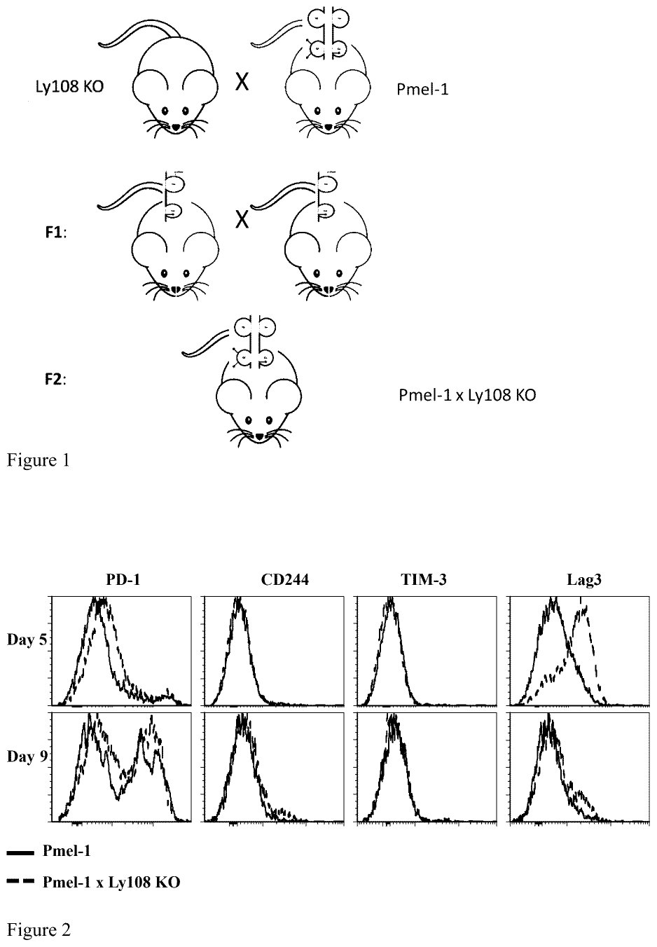

In another embodiment the invention relates to a transgenic mouse model herein designated Pmel×Ly108KO. As disclosed herein for the first time, Pmel×Ly108KO mice are characterized by CD8+ T cells lacking Ly108 (the murine homologue of human SLAMF6) and expressing a transgenic TCR against H-2Db gp100: 25-33 peptide (recognizing the murine homologue of human premelanosome protein, which is often overexpressed in human melanomas). As further disclosed herein, these mice may be generated by breeding Pmel-1 mice with Ly108−/− mice, as illustrated in FIG. 1.

The following examples are presented in order to more fully illustrate some embodiments of the invention. They should, in no way be construed, however, as limiting the broad scope of the invention.

### EXAMPLES

### Materials and Methods

Antibodies: For flow cytometry, cells were labeled with the following reagents: anti-TIM3 (RMT3-23), and anti-LAG-3 (C9B7W) (all from Biolegend, San Diego, CA, USA). anti-PD1 (J43), and anti-CD244 (eBio244F4) were from eBioscience. Anti-LAG-3 (C9B7W) and the corresponding isotype were from InVivoMab, BioXcell, NH, USA.

Splenocyte activation: Pmel-1 or Pmel-1×SLAMF6−/− (Pmel-1×Ly108 KO) mouse splenocytes (2×106/ml) were activated with 1 μg/ml of mouse gp10025-33 peptide for 6 days with IL-2 30 IU/ml. Fresh medium containing IL-2 was added every other day.

Co-culture of T cells with splenocytes: Mouse CD8+ T cells were separated using Mouse CD8+ T cell isolation kit (EasySep 19853A, Stemcell Technologies, Vancouver, CA) and 1×105 cells were co-cultured overnight at the indicated ratios with non-T-splenocytes. Conditioned medium was collected, and mouse IFN-γ secretion was detected by ELISA (Biolegend).

Interferon-gamma secretion: Mouse splenocytes, previously activated for 7 days, were co-cultured (1×105) overnight at a 1:1 ratio with the indicated target cells or activated with 1 μg/ml plate-bound anti-CD3 (Biolegend, clone: 145-2C11) as indicated in each experiment. Conditioned medium was collected, and mouse IFN-γ secretion was detected by ELISA (Biolegend).

Flow cytometry. After blocking Fc receptors with anti-CD16/CD32 antibody, cells were stained with antibodies or tetramers on ice or at room temperature for 25 min, according to the manufacturer's instructions. Subsequently, cells were washed and analyzed using CytoFlex (Beckman Coulter, CA, USA), and flow cytometry-based sorting was done in an ARIA-III sorter. Flow cytometry analysis was done using FCS express 5 flow research edition (De Novo software).

In vivo assays: B16-F10/mhgp100 mouse melanoma cells (0.5×106) were injected s.c. into the back of C57BL/6 mice. Pmel-1×SLAMF6−/− mouse splenocytes (2×106/ml) were expanded with 1 μg/ml of the gp10025-33 peptide in the presence of IL-2 (30 IU/ml) and either anti-LAG3 Ab (10 g/ml) or the corresponding isotype. Fresh medium containing IL-2 and the Ab was added every other day. On day 7, 107 Pmel-1×SLAMF6−/− cells activated in the presence of anti-LAG3 or isotype were adoptively transferred i.v. into 500 CGy-irradiated tumor-bearing mice. 0.25×106 IU/100p IL-2 was administered i.p. twice a day for 2 days. 100 g anti-LAG3 or isotype was administered i.p. in 100 μl PBS 5 times during the 2 weeks post-adoptive transfer. Tumor size and mouse weight were measured 3 times a week. Follow up was conducted until day 30.

For the wild-type SLAMF6 control experiment, Pmel-1 splenocytes (expressing native SLAMF6) were used. To this end, B16-F10/mhgp100 mouse melanoma cells (0.5×106) were injected s.c. into the back of C57BL/6 mice. Pmel-1 mouse splenocytes (2×106/ml) were expanded with 1 μg/ml of the gp10025-33 peptide in the presence of IL-2 (30 IU/ml). Fresh medium containing IL-2 was added every other day. On day 7, 107 Pmel-1 cells were adoptively transferred i.v. into 500 CGy-irradiated tumor-bearing mice. 100 g anti-LAG3 was administered i.p. in 100 μl PBS 5 times during the 2 weeks post-adoptive transfer. Tumor size and mouse weight were measured 3 times a week. Follow up was conducted until day 58.

Combination therapy with soluble SLAMF6 ectodomain polypeptide and anti-LAG3 antibody: Pmel-mouse splenocytes (2×106/ml) were activated with 1 μg/ml of mouse gp10025-33 peptide for 7 days with either a soluble SLAMF6 ectodomain polypeptide corresponding to SLAMF6var3 (seSLAMF6 V3, 50 μg/ml), an anti LAG3 antibody (anti Lag3, 10 μg/ml) or a combination thereof. Fresh medium containing reagents was added every other day. At day 7, activated splenocyte were co-cultured (1×105) overnight at a 1:1 ratio with B16-F10/mhgp100 mouse melanoma cells. Conditioned medium was collected, and mouse IFN-γ secretion was detected by ELISA (Biolegend).

The sequence of the SLAMF6var3 ectodomain polypeptide used in the experiment is presented in SEQ ID NO: 8 below, and includes an N′ signal peptide (bold) and a C′ 6-histidine tag, as follows:

(SEQ ID NO: 8)
MLWLFQSLLFVFCFGPGNVVSVPHETKSPEIHVTNPKQGKRLNFTQSYS
LQLSNLKMEDTGSYRAQISTKTSAKLSSYTLRILRQLRNIQVTNHSQLF
QNMTCELHLTCSVEDADDNVSFRWEALGNTLSSQPNLTVSWDPRISSEQ
DYTCIAENAVSNLSFSVSAQKLCEDVKIQYTDTKMGSHHHHHH.

Statistics. Statistical significance was determined by unpaired t-test (two-tailed with equal SD) using Prism software (GraphPad). A p-value<0.05 was considered statistically significant. Analysis of more than two groups was performed using the one-way ANOVA test. Unless indicated otherwise, *, p≤0.05; **, p≤0.01; ***, p≤0.001. For each experiment, the number of replicates performed and the statistical test used are stated in the corresponding figure legend.

### Example 1. SLAMF6 Knockout Mice

In order to evaluate the role of SLAMF6 in melanoma-cognate T cells, the following mouse strains were used and generated (see FIG. 1).

Ly108 knockout mice (Ly108−/−, Ly108KO)

Ly108 is the murine homologue of human SLAMF6. Ly108−/− (Ly108 knockout, Ly108 KO) mice have a STOP codon inserted into exon 2, and a neo cassette replacing exon 3 and part of exon 2 of the Slamf6 gene, abolishing gene expression. The mice were generated from the HGTC-8 C57BL/6J ES stem cell line and do not express Ly108, as detected by flow cytometry and immunoblotting (Dutta M; Schwartzberg P L. 2012. Characterization of Ly108 in the thymus: evidence for distinct properties of a novel form of Ly108. J Immunol 188(7):3031-41PubMed: 22393150MGI: J:183088).

### Pmel-1 Transgenic Mice

This transgenic strain carries a rearranged T cell receptor transgene specific for the murine homologue (pmelSi or pmel-17) of human premelanosome protein (referred to as PMEL, SILV or gp100), and the T lymphocyte specific Thy1a (Thy1.1) allele (Overwijk W W; Restifo N P. 2003. Tumor regression and autoimmunity after reversal of a functionally tolerant state of self-reactive CD8+ T cells. J Exp Med 198(4):569-80PubMed: 12925674MGI: J:85058). The pmel-1 mouse was developed as a model system for studying the treatment of malignant melanoma using adoptive cell therapy. The target antigen, pmel-17, is an ortholog of the melanocyte differentiation antigen gp100, which is often overexpressed in human melanomas.

### Generation of Pmel×Ly108KO Mice

This mouse strain was generated by breeding Pmel−1 mice with Ly108−/− mice, as illustrated in FIG. 1.

In order to select homozygous mice of both genes, weened F2 mice were subjected to genotyping by PCR reaction to the Ly108 locus on chromosome 1 (primers adapted from Jackson laboratories website) and in the Pmel-1 locus on chromosome 2 (primers described previously (Ref: Ji, Yun, et al. Identification of the genomic insertion site of Pmel-1 TCR a and R transgenes by next-generation sequencing. PloS one 9 e96650 (2014)).

The new mouse strain serves a source for CD8+ T cells lacking Ly108 and expressing the transgenic TCR against H-2Db gp100: 25-33 peptide.

### Example 2. SLAMF6 Down-Regulation is Associated with Selective Up-Regulation of LAG3 Expression

Pmel-1 and Pmel-1×Ly108 KO splenocytes (2×106/ml) were expanded with 1 μg/ml of the gp10025-33 peptide (EGSRNQDWL, SEQ ID NO: 9) in the presence of 30 IU/ml IL-2 for 7 days in RPMI1640 culture medium supplemented with 10% heat-inactivated FCS, 2 mmol/L L-glutamine, 50 μM βME, 1% Non-essential amino acids, 1% Sodium Pyruvate and combined antibiotics (all from Invitrogen Life Technologies).

After the expansion phase, the cells were kept for additional 5 or 9 days in culture medium without the addition of IL-2. Surface expression of selected exhaustion markers was measured by flow-cytometry. The following anti mouse antibodies were used for the analysis: anti-PD-1 (eBioscience, ref: 47-9985-82), anti-CD244/SLAMF4 (eBioscience, ref: 12-2441-82), anti-LAG3 (Biolegend, clone: C9B7W, cat: 125208) and anti-TIM-3 (Biolegend, cat: 119704), all at concentration of 0.2 g per 1001 staining reaction.

As shown in FIG. 2, the surface expression of PD-1, CD244 and TIM-3 on Pmel-1×Ly108KO splenocytes was similar to their expression on Pmel-1 splenocytes at 5 and 9 days following the 7-day activation and expansion period. However, the expression of LAG3 surprisingly differed between the two cell populations. Pmel-1×Ly108KO splenocytes exhibited marked elevation in their surface expression of LAG3 at 5 days post-activation, compared to Ly108-expressing Pmel-1 cells.

These results demonstrate that SLAMF6 down-regulation is associated with selective up-regulation of LAG3 expression on lymphocytes following prolonged antigen-induced activation. In contradistinction, the expression of other immune checkpoint inhibitors such as PD-1, CD244 and TIM-3 remained substantially unaltered despite the down-regulation of SLAMF6.

### Example 3. Synergistic Effect of SLAMF6 and LAG3 in Combination in Augmenting Tumor-Specific T Cell Response

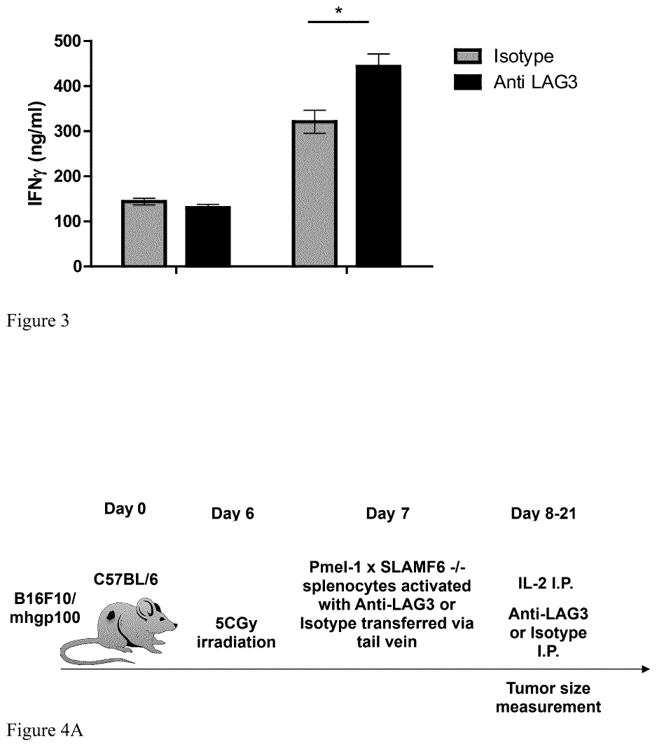

Pmel-1 and Pmel-1×Ly108 KO splenocytes were expanded for 7 days as described in Example 1. Where indicated in FIG. 3, the expansion was performed with the addition of 10 g/ml anti-LAG3 blocking antibody (BioXCell, clone: C9B7W, cat: BE0174; “anti-LAG3”) or an isotype control antibody (BioXCell, cat: BE0088; “isotype”). After 7 days of expansion, the cells were washed and co-cultured overnight with B16-F10/mhgp100 melanoma cells at a 1:1 effector: target ratio. Interferon gamma (IFN-γ) secretion was measured by ELISA (Biolegend).

As can be seen in FIG. 3, activation in the presence of LAG3 blocking antibodies significantly improved IFN-γ secretion from Pmel-1×Ly108 KO cells incubated with cognate melanoma cells (FIG. 3, right two histograms), while no effect on Ly108-expressing Pmel-1 cells was measured (FIG. 3, left two histograms). Surprisingly, Ly108 down-regulation together with LAG3 blockade resulted in a 3-times higher IFN-γ production compared to the Ly108-expressing cells (either with or without LAG3 blockade).

Overall, the results demonstrate a synergistic effect for the combination of SLAMF6 and LAG3 in augmenting tumor-specific T cell response. Without wishing to be bound by a specific theory or mechanism of action, LAG3 may represent a compensatory mechanism for the enhanced activation of SLAMF6 knockout T cells. Modulating both SLAMF6 and LAG3 expression or activity may abrogate this compensatory mechanism, thereby providing unexpectedly improved anti-tumor response compared to other therapies, such as conventional immunotherapy approaches directed at immune checkpoint molecules.

### Example 4. Improved Anti-Tumor Efficacy In Vivo

To evaluate the combination of SLAMF6−/− cells and LAG3 blocking antibody in vivo, an adoptive cell transfer (ACT) experiment in melanoma bearing mice, using Pmel-1×SLAMF6−/− CD8+ T cells activated in vitro in the presence of anti-LAG3 and sustained by intraperitoneal IL-2 (day 8 and 9) and anti-LAG-3 (days 8, 10, 15, 18, 21). The control arm consisted of Pmel-1×SLAMF6−/− CD8+ T cells stimulated and sustained with an isotype antibody.

To this end, B16-F10/mhgp100 mouse melanoma cells were injected s.c. into the back of C57BL/6 mice. Pmel-1×SLAMF6−/− (Pmel-1×Ly108 KO) mouse splenocytes were expanded with gp100 25-33 peptide and IL-2 (30 IU/ml) in the presence of either Anti-Lag3 or Isotype control.

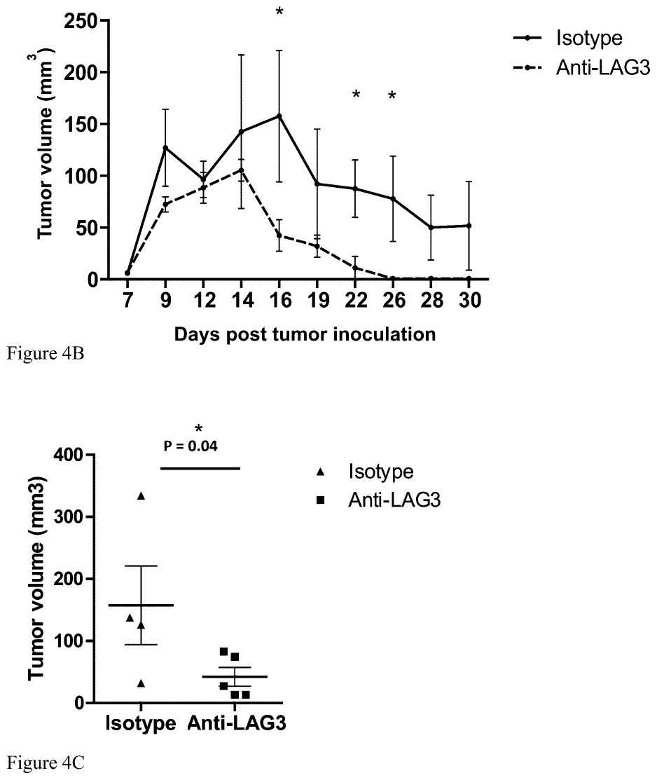

On day 7, Isotype or Anti-Lag3 activated cells were adoptively transferred i.v. into the irradiated tumor-bearing mice. Anti-LAG3 or Isotype control antibodies were injected i.p. 5 times in the 2 weeks post transfer. N=5 mice per group. Tumor size was measured 3 times a week. A scheme of the experimental layout is provided in FIG. 4A, and the results are presented in FIGS. 4B-4C, depicting the tumor volume until day 30 post tumor inoculation or on day 16 post tumor inoculation, respectively.

As can be seen in FIGS. 4B-4C, mice treated by the combination of SLAMF6 deficient T cells and anti-LAG3 antibody showed faster reduction and disappearance of their tumors (FIG. 4B), apparent already on day 16 (p=0.04) (FIG. 4C). For example, on day 16, the tumors at the combination group were about 3-fold smaller in volume than the tumors treated by SLAMF6 deficient T cells alone, about 10-fold smaller on day 22, and up to over 100-fold smaller thereafter, until the tumors at the combination group were non-measurable as of day 26.

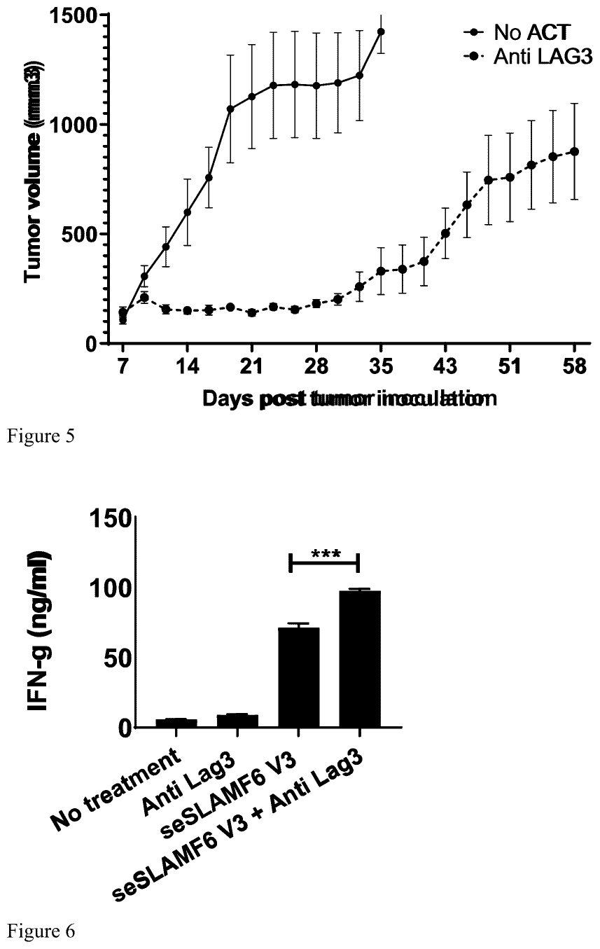

In contradistinction, as can be seen in FIG. 5, in an equivalent experiment performed with Pmel-1 splenocytes (expressing native SLAMF6), a temporary delay in tumor development was evident when systemic administration of anti-LAG3 antibodies was used to supplement the adoptive transfer therapy; however, no tumor regression was demonstrated, and tumor growth resumed as of the fifth week post tumor inoculation.

These results demonstrate that blocking the compensatory rise of LAG3 on SLAMF6−/− T cells markedly improved their anti-tumor effect and exhibited therapeutic synergy in vivo.

### Example 5. Synergistic Effect of a Combination Treatment with a SLAMF6-Mediated T Cell Activator and a LAG3 Inhibitor on Tumor-Specific T Cell Response

Pmel-1 splenocyte were activated for 7 days with their specific peptide, essentially as described in Example 3, in the presence of the following treatments: co-culturing with a soluble SLAMF6 ectodomain polypeptide corresponding to SLAMF6var3 (seSLAMF6 V3, 50 μg/ml), with an anti LAG3 antibody (anti Lag3, 10 μg/ml), with a combination of these agents (seSLAMF6 V3+anti Lag3, 50 and 10 μg/ml respectively), or without any supplementation (No treatment). Following activation, cells were co-cultured with cognate melanoma cells and IFN-γ secretion was measured, essentially as described in Example 3.

As can be seen in FIG. 6, IFN-γ secretion was 1.5-fold higher in the anti-Lag3 treatment group compared to control, and 11-fold higher in the seSLAMF6 V3 treatment group. Surprisingly, the seSLAMF6 V3+anti-Lag3 group, having received both modulators in combination, exhibited a 16-fold enhancement in tumor-induced IFN-γ secretion, which significantly (p=0.0002) exceeded the projected combined level based on the values measured in each treatment when given alone. Remarkably, the secreted IFN-γ level measured following combination therapy was over 10-fold higher compared to anti-LAG3 alone, and about 1.5-fold higher compared to seSLAMF6 V3 alone. These results further demonstrate the advantageous use of SLAMF6-mediated T cell activators and LAG3 inhibitors in combination, including soluble polypeptides and antibodies, achieving improved, synergistic anti-tumor activity.

### Example 6. Evaluation of In Vivo Efficacy Against Multiple Tumor Types Using Genetically Engineered Human Lymphocytes

NY-ESO-1 (also known as cancer/testis antigen 1, LAGE2 or LAGE2B) is a tumor-associated antigen expressed on many solid tumors, including, but not limited to, synovial sarcoma, melanoma, multiple myeloma, neuroblastoma and carcinomas of lung, esophagus, liver, gastrointestinal system, prostate, ovary, breast and bladder. Global data indicates that in the majority of tumors, NY-ESO-1 is frequently expressed in metastatic, high grade/advanced stage tumors, and is as such associated with poor prognosis. A number of pre-clinical studies and clinical trials (completed and ongoing) explore the potential efficacy of immunotherapeutic strategies against NY-ESO-1 expressing tumors.

An adoptive transfer therapy model for examining the efficacy of the synergistic combinations of the invention with human lymphocytes genetically engineered to express a TCR directed against NY-ESO-1, is conducted in NSG mice.

On day 0 of the experiment, mice are injected subcutaneously with 1×106 NY-ESO-1—expressing tumor cells (selected from the group consisting of: synovial sarcoma, melanoma multiple myeloma, neuroblastoma and carcinomas of lung, esophagus, liver, gastrointestinal system, prostate, ovary, breast and bladder), mixed with Matrigel. On the same day, human T cells (lymphocytes) are thawed and activated for 2 days with an anti CD3 antibody (OKT3, 50 ng/ml) and cultured in culture medium (CM) supplemented with either IL-2, a SLAMF6-mediated T cell activator (including soluble SLAMF6 ectodomain polypeptides at a concentration ranging from 10 to 300 μg/ml), a LAG3 inhibitor (including anti-LAG3 antibodies at a concentration ranging from 1 to 100 μg/ml) or a combination thereof.

On day 2, lymphocytes are transduced with a vector expressing a TCR directed against NY-ESO-1 collected from producer cells. On day 3 lymphocytes are transferred in culture and grown, and on day 6 a sample is stained with a marker to verify transduction efficiency. On day 7, the transduced lymphocytes are injected i.v. to the mice at cell numbers ranging from 1 to 20×106 cells per mouse, and tumor development is monitored.

Between days 7-14, mice are injected every other day with IL-2 (60,000 U) or the various tested treatments: SLAMF6-mediated T cell activator (100-500 μg per injection), LAG3 inhibitor (100-500 g per injection) or a combination thereof.

Mice are monitored every two days for weight, general physical condition and tumor volume (by caliper). Mice are sacrificed when tumor volume reaches 1500 mm3.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of treating a tumor, or of enhancing an anti-tumor immune response, in a subject in need thereof, comprising administering to the subject therapeutically effective amounts of a SLAMF6-mediated T cell activator and a LAG3 inhibitor in concurrent or sequential combination, wherein the SLAMF6-mediated T cell activator is selected from the group consisting of soluble SLAMF6 ectodomain polypeptides, anti-SLAMF6 antibodies, and SLAMF6-binding fragments thereof, and wherein the LAG3 inhibitor is selected from the group consisting of soluble LAG3 polypeptides, anti-LAG3 antibodies, and LAG3-binding fragments thereof.

2. The method of claim 1, wherein the SLAMF6-mediated T cell activator is selected from the group consisting of soluble SLAMF6 ectodomain polypeptides and anti-SLAMF6 antibodies, and wherein the LAG3 inhibitor is selected from the group consisting of soluble LAG3 polypeptides and anti-LAG3 antibodies.

3. The method of claim 1, wherein the tumor is selected from the group consisting of tumors of the skin, urinary tract, head and neck, brain, bladder, liver, lung, breast, reproductive organs, prostate, intestinal tract, bone, and connective tissues.

4. The method of claim 3, wherein said tumor is a solid tumor positive for one or more molecular markers selected from the group consisting of MHC class II, LAG3, CD4 and CD8.

5. The method of claim 3, wherein said tumor is melanoma.

6. The method of claim 3, wherein said tumor is resistant to immune checkpoint inhibitor therapy selected from the group consisting of LAG3 inhibitors, PD-1 inhibitors, PD-L1 inhibitors, CTLA4 inhibitors, and combinations thereof.

7. The method of claim 3, wherein the SLAMF6-mediated T cell activator and a LAG3 inhibitor are administered in concurrent or sequential combination with adoptive transfer cell therapy.

8. The method of claim 7, wherein said SLAMF6-mediated T cell activator and said LAG3 inhibitor are administered to the subject for 3-14 days prior to administering said subject with the adoptive transfer cell therapy.

9. The method of claim 3, wherein the subject is not under treatment regimen with other immune checkpoint inhibitors.

10. The method of claim 3, for enhancing a T-cell mediated anti-tumor immune response in said subject.

11. The method of claim 2, wherein said SLAMF6-mediated T cell activator is a polypeptide comprising an isolated human SLAMF6 ectodomain, a T-cell stimulating antibody having binding specificity thereto, or an antigen-binding fragment thereof, and wherein said LAG3 inhibitor is a LAG3-specific neutralizing antibody, an antigen-binding fragment thereof, or a polypeptide comprising the extracellular domain of human LAG3.

12. A method of treating a tumor, or of enhancing an anti-tumor immune response in a subject in need thereof, comprising administering to the subject a cell composition adapted for adoptive transfer immunotherapy, prepared by a method comprising the step of expanding an immune cell population in the presence of a SLAMF6-mediated T cell activator and a LAG3 inhibitor, wherein the SLAMF6-mediated T cell activator is selected from the group consisting of soluble SLAMF6 ectodomain polypeptides, anti-SLAMF6 antibodies, and SLAMF6-binding fragments thereof, and wherein the LAG3 inhibitor is selected from the group consisting of soluble LAG3 polypeptides, anti-LAG3 antibodies, and LAG3-binding fragments thereof.

13. The method of claim 12, wherein the cell population contains T cells, NK cells or combinations thereof, wherein the SLAMF6-mediated T cell activator is selected from the group consisting of soluble SLAMF6 ectodomain polypeptides, and anti-SLAMF6 antibodies, and wherein the LAG3 inhibitor is selected from the group consisting of soluble LAG3 polypeptides and anti-LAG3 antibodies.

14. The method of claim 13, wherein the cell composition adapted for adoptive transfer is histocompatible with the subject, or wherein the cell composition adapted for adoptive transfer is autologous to the subject.

15. The method of claim 13, wherein the tumor is selected from the group consisting of tumors of the skin, urinary tract, head and neck, brain, bladder, liver, lung, breast, reproductive organs, prostate, intestinal tract, bone, and connective tissues.

16. The method of claim 13, wherein said tumor is melanoma or is resistant to immune checkpoint inhibitor therapy selected from the group consisting of LAG3 inhibitors, PD-1 inhibitors, PD-L1 inhibitors, CTLA4 inhibitors, and combinations thereof.

17. The method of claim 13, wherein the tumor is a solid tumor positive for one or more molecular markers selected from the group consisting of MHC class II, LAG3, CD4 and CD8.

18. The method of claim 13, further comprising administering a SLAMF6-mediated T cell activator and a LAG3 inhibitor in concurrent or sequential combination with the cell composition adapted for adoptive transfer, or wherein the subject is not under treatment regimen with other immune checkpoint inhibitors.

19. The method of claim 12, wherein said SLAMF6-mediated T cell activator is a polypeptide comprising an isolated human SLAMF6 ectodomain, a T-cell stimulating antibody having binding specificity thereto, or an antigen-binding fragment thereof, and wherein said LAG3 inhibitor is a LAG3-specific neutralizing antibody, an antigen-binding fragment thereof, or a polypeptide comprising the extracellular domain of human LAG3.
